SEQUENCE LISTING (5 sequences)

SEQ ID NO: 1 - protein, 4 aa, Artificial
  Synthetic peptide.
KRDS

SEQ ID NO: 2 - protein, 4 aa, Artificial
  Synthetic peptide.
KRES

SEQ ID NO: 3 - protein, 4 aa, Artificial
  Synthetic peptide.
KERS

SEQ ID NO: 4 - protein, 4 aa, Artificial
  Synthetic peptide.
KRES

SEQ ID NO: 5 - protein, 4 aa, Artificial
  Synthetic peptide.
FREL

(19) United States
(12) Patent Application Publication
(10) Pub. No.: US 2006/0173067 A1
(43) Pub. Date: Aug. 3, 2006

(54) Small molecules for the treatment of atherosclerosis

(75) Inventors: Alan M. Fogelman, Beverly Hills, CA (US); Gattadahalli M. Anantharamaiah, Birmingham, AL (US); Mohamad Navab, Los Angeles, CA (US)

Correspondence Address: QUINE INTELLECTUAL PROPERTY LAW GROUP, P.C., P O BOX 458, ALAMEDA, CA, 94501, US

(73) Assignees: The Regents of the University of California; The University of Alabama Research Foundation

(21) Appl. No.: 11/201,004

(22) Filed: Aug. 9, 2005

Related U.S. Application Data
(60) Provisional application No. 60/600,925, filed on Aug. 11, 2004.

(51) Int. Cl. A61K 31/401; A61K 31/366; A61K 31/22; A61K 31/198; C07C 229/02

(52) U.S. Cl. 514/423; 514/567; 514/460; 514/548; 554/116

(57) ABSTRACT

This invention provides novel small molecules that ameliorate one or more symptoms of atherosclerosis. The small molecules are highly stable and readily administered via an oral route. The small molecules are effective to stimulate the formation and cycling of pre-beta high density lipoprotein-like particles and/or to promote lipid transport and detoxification. This invention also provides a method of tracking a small molecule in a mammal. In addition, the small molecules inhibit osteoporosis. When administered with a statin, the small molecules enhance the activity of the statin permitting the statin to be used at significantly lower dosages and/or cause the statins to be significantly more anti-inflammatory at any given dose.

### CROSS-REFERENCE TO RELATED APPLICATIONS

This application claims benefit of and priority to U.S. Ser. No. 60/600,925, filed on Aug. 11, 2004, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported by United States Public Health Service and National Heart, Lung, and Blood Institute Grants HL30568 and HL34343. The Government of the United States of America may have certain rights in this invention.

### FIELD OF THE INVENTION

This invention relates to the field of atherosclerosis. In particular, this invention pertains to the identification of a class of peptides that are orally administrable and that ameliorate one or more symptoms of atherosclerosis.

### BACKGROUND OF THE INVENTION

Cardiovascular disease is a leading cause of morbidity and mortality, particularly in the United States and in Western European countries. Several causative factors are implicated in the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesteremia (high blood cholesterol concentrations) provide a significant risk factor associated with atherosclerosis.

Cholesterol is present in the blood as free and esterified cholesterol within lipoprotein particles, commonly known as chylomicrons, very low density lipoproteins (VLDLs), low density lipoproteins (LDLs), and high density lipoproteins (HDLs). Concentration of total cholesterol in the blood is influenced by (1) absorption of cholesterol from the digestive tract, (2) synthesis of cholesterol from dietary constituents such as carbohydrates, proteins, fats and ethanol, and (3) removal of cholesterol from blood by tissues, especially the liver, and subsequent conversion of the cholesterol to bile acids, steroid hormones, and biliary cholesterol.

Maintenance of blood cholesterol concentrations is influenced by both genetic and environmental factors. Genetic factors include concentration of rate-limiting enzymes in cholesterol biosynthesis, concentration of receptors for low density lipoproteins in the liver, concentration of rate-limiting enzymes for conversion of cholesterols bile acids, rates of synthesis and secretion of lipoproteins and gender of person. Environmental factors influencing the hemostasis of blood cholesterol concentration in humans include dietary composition, incidence of smoking, physical activity, and use of a variety of pharmaceutical agents. Dietary variables include amount and type of fat (saturated and polyunsaturated fatty acids), amount of cholesterol, amount and type of fiber, and perhaps amounts of vitamins such as vitamin C and D and minerals such as calcium.

The main stay for the prevention and treatment of atherosclerosis has been the use of statins, which lower plasma levels of low density lipoproteins (LDL). Recently, there has been an increased awareness of the potential for HDL-based therapies to prevent and treat atherosclerosis. The intravenous infusion of apolipoprotein A-I_{Milano} was shown to rapidly reduce coronary artery plaque (Nissen et al. (2003) JAMA, 290: 2292-2300; Rader (2003) JAMA 290: 2322-2324.). This therapy, however, requires a recombinant protein containing 243 amino acids with a molecular weight of approximately 28,000 Daltons that must be given intravenously. We have previously described a series of 18 D-amino acid peptides with molecular weights on the order of 2,400 Daltons that mimic many of the properties of the main protein in HDL, apoA-I, which has a molecular weight of 28,000 Daltons (see, e.g., U.S. Pat. No. 6,664,230, and PCT Applications WO 02/15923 and WO 2004/034977). These 18 D-amino acid peptides can be given orally and dramatically reduce atherosclerosis in mice without significantly altering plasma cholesterol levels (Navab et al. (2002) Circulation, 105: 290-292; Navab et al. (2004) J Lipid Res, 45: 993-1007; Navab et al. (2004) Circulation, In press). When given orally these 18 amino acid peptides result in reduced plasma and lipoprotein lipid hydroperoxides in mice (Navab et al. (2004) Circulation, In press) and monkeys (Navab et al. (2004) J Lipid Res, 45:993-1007). They also convert pro-inflammatory HDL (HDL that promotes LDL-induced monocyte chemotactic activity in a human artery wall coculture) to anti-inflammatory HDL (HDL that decreases LDL-induced monocyte chemotactic activity) in mice (Navab et al. (2002) Circulation, 105: 290-292; Navab et al. (2004) Circulation, In press) and monkeys (Navab et al. (2004) J Lipid Res, 45:993-1007). When given orally, they also decreased the ability of LDL to induce human artery wall cells to produce monocyte chemotactic activity (Navab et al. (2002) Circulation, 105: 290-292; Navab et al. (2004) J Lipid Res, 45: 993-1007; Navab et al. (2004) Circulation, In press). Additionally, these 18 amino acid peptides promoted cholesterol efflux from macrophages after oral administration to mice (Navab et al. (2004) Circulation, In press) and monkeys (Navab et al. (2004) J Lipid Res, 45:993-1007).

### SUMMARY OF THE INVENTION

This invention provides novel small organic molecules (e.g., MW less than about 900 Da) administration of which mitigates one or more symptoms of atherosclerosis and/or other pathologies characterized by an inflammatory response. Such conditions include, but are not limited to rheumatoid arthritis, lupus erythematous, polyarteritis nodosa, hronic obstructive pulmonary disease (asthma), diabetes, osteoporosis, Alzheimer's disease, congestive heart failure, endothelial dysfunction, viral illnesses such as influenza A, and diseases such as multiple sclerosis. In addition, the molecules appear effective in mitigating one or more symptoms associated with diabetes and/or asthma. The small organic molecules can be administered by any of a variety of modalities, but it is noted, in particular that they arte suitable for oral administration and when so administered, are readily taken up and delivered to the serum, and are effective to mitigate one or more symptoms of atherosclerosis

In certain embodiments, The small organic molecules of this invention are typically effective to stimulate the formation and cycling of pre-beta high density lipoprotein-like particles and/or to promote lipid transport and detoxification.

In certain embodiments, The small organic molecules described herein are also effective for preventing the onset or inhibiting or eliminating one or more symptoms of osteoporosis.

In certain embodiments, the small organic molecules can be used to enhance (e.g. synergically enhance) the activity of statins and/or Ezetimibe or other cholesterol uptake inhibitors, thereby permitting the effective use of statins or cholesterol uptake inhibitors at lower dosages and/or cause the statins or cholesterol uptake inhibitors to be significantly more anti-inflammatory at any given dose.

Thus, in certain embodiments, this invention provides small organic molecules or a combination of small organic molecules and/or peptides that ameliorates one or more symptoms of an inflammatory condition (e.g., atherosclerosis atherosclerosis, rheumatoid arthritis, lupus erythematous, polyarteritis nodosa, osteoporosis, chronic obstructive pulmonary disease (asthma), diabetes, Altzheimer's disease, a viral illnesses, asthma, diabetes, etc.). Certain preferred small organic molecules are soluble in ethyl acetate at a concentration greater than about 4 mg/mL; are soluble in aqueous buffer at pH 7.0; and/or when contacted with a phospholipid in an aqueous environment, forms particles, or participate in the formation of particles with a diameter of approximately 7.5 nm and/or form or participate in the formation of stacked bilayers with a bilayer dimension on the order of 3.4 to 4.1 nm with spacing between the bilayers in the stack of approximately 2 nm. Typically the small organic molecules of this invention have a molecular weight less than about 900 daltons. In certain embodiments, the small organic molecules convert pro-inflammatory HDL to anti-inflammatory HDL or makes anti-inflammatory HDL more anti-inflammatory.

In certain embodiments, these small organic molecules protect a phospholipid (e.g., 1-palmitoyl-2-arachidonoyl-sn-glycero-3-phosphorylcholine (PAPC), 1-stearoyl-2-arachidonoyl-sn-glycero-3-phosphorylcholine (SAPC)), and 1-stearoyl-2-arachidonyl-sn-glycero-3-phosphorylethanolamine (SAPE). In certain embodiments, these small organic molecules can include, but need not be limited to any of the small organic molecules described herein.

In certain embodiments, the small organic molecules of this invention are not analogues of the amino acid sequence Lys-Arg-Asp-Ser (SEQ ID NO:1) in which Lys, Arg, Asp, and Ser are all L amino acids.

This invention also provides pharmaceutical formulations comprising one or more of the small organic molecules described herein and/or one or more of the peptides described in U.S. Ser. No. 10/649,378, and a pharmaceutically acceptable excipient. Typically the small organic molecule (s) are present in an effective dose. The small organic molecule (s) can also be provided as a time release formulation and/or as a unit dosage formulation. In certain embodiments, the formulation is formulated for oral administration. In certain embodiments, the formulation is formulated for administration by a route selected from the group consisting of oral administration, inhalation (e.g., nasal administration, oral inhalation, etc.), rectal administration, intraperitoneal injection, intravascular injection, subcutaneous injection, transcutaneous administration, inhalation administration, intramuscular injection, and the like.

Also provided is a kit comprising a container containing one or more of the small organic molecule (s) described herein and instructional materials teaching the use of the small organic molecule (s) in the treatment of a pathology characterized by an inflammatory response (e.g., atherosclerosis atherosclerosis, rheumatoid arthritis, lupus erythematous, polyarteritis nodosa, asthma, osteoporosis, Altzheimer's disease, a viral illnesses, etc.).

This invention also provides a method of mitigating (e.g., reducing or eliminating) one or more symptoms of atherosclerosis in a mammal (human or non-human mammal). The method typically involves administering to the mammal an effective amount of one or more of the small organic molecule(s) described herein and/or one or more of the peptides described in U.S. Ser. No. 10/649,378. The small organic molecules can be administered in a in a pharmaceutically acceptable excipient (e.g., for oral administration, etc.) and can, optionally be administered in conjunction (e.g., before, after, or simultaneously) with a lipid. The administering can comprise administering the small organic molecule by a route selected from the group consisting of oral administration, inhalation, rectal administration, intraperitoneal injection, intravascular injection, subcutaneous injection, transcutaneous administration, intramuscular injection, and the like. In certain embodiments, the mammal is a mammal diagnosed as having one or more symptoms of atherosclerosis. In certain embodiments, the mammal is a mammal diagnosed as at risk for stroke or atherosclerosis.

In another embodiment, this invention provides method of mitigating one or more symptoms of an inflammatory pathology (e.g., atherosclerosis, rheumatoid arthritis, lupus erythematous, polyarteritis nodosa, osteoporosis, multiple sclerosis, diabetes, asthma, Altzheimer's disease, a viral illnesses, etc.). The method typically involves administering to the mammal an effective amount of one or more of the small organic molecules described herein. The small organic molecule(s) can be administered in a in a pharmaceutically acceptable excipient (e.g., for oral administration) and can, optionally be administered in conjunction (e.g., before, after, or simultaneously) with a lipid. The administering can comprise administering the small organic molecules by a route selected from the group consisting of oral administration, inhalation administration, rectal administration, intraperitoneal injection, intravascular injection, subcutaneous injection, transcutaneous administration, and intramuscular injection. In certain embodiments, the mammal is a mammal diagnosed as having one or more symptoms of of the inflammatory pathology. In certain embodiments, the mammal is a mammal diagnosed as at risk for the inflammatory pathology.

The small organic molecules of this invention also act synergistically with statins and/or with a selective cholesterol uptake inhibitor (e.g., Ezetimibe). The method typically involves coadministering with the statin and/or cholesterol uptake inhibitor an effective amount of one or more of the small organic molecules described herein. In certain embodiments, the statin is selected from the group consisting of cerivastatin, atorvastatin, simvastatin, pravastatin, fluvastatin, lovastatin. rosuvastatin, and pitavastatin. The small organic molecules can be administered before, after, or simultaneously with the statin and/or the cholesterol uptake inhibitor. The small organic molecules and/or said statin and/or cholesterol uptake inhibitor can be administered as a unit dosage formulation. In certain embodiments, the administering comprises administering the small organic molecules and/or the statin by a route selected from the group consisting of oral administration, inhalation administration, rectal administration, intraperitoneal injection, intravascular injection, subcutaneous injection, transcutaneous administration, intramuscular injection, and the like. The mammal includes, but is not limited to a mammal diagnosed as having one or more symptoms of atherosclerosis or diagnosed as at risk for stroke or atherosclerosis.

This invention also provides a method of mitigating one or more symptoms associated with atherosclerosis in a mammal. The method typically involves administering a statin and/or a selective cholesterol uptake inhibitor; and an effective amount of one or more small organic molecules described herein, where the the effective amount of the statin and/or cholesterol uptake inhibitor is lower than the effective amount of a statin or a cholesterol uptake inhibitor administered without the small organic molecule(s). In certain embodiments, the effective amount of the small organic molecule(s) is lower than the effective amount of the small organic molecules administered without the statin and/or cholesterol uptake inhibitor. In certain embodiments, the statin is selected from the group consisting of cerivastatin, atorvastatin, simvastatin, pravastatin, fluvastatin, lovastatin. rosuvastatin, and pitavastatin. The small organic molecule can be administered before, after, or simultaneously with the statin and/or the cholesterol uptake inhibitor. The small organic molecule and/or the statin and/or cholesterol uptake inhibitor can be administered as a unit dosage formulation. In certain embodiments, the administering comprises administering the small organic molecules and/or the statin by a route selected from the group consisting of oral administration, inhalation administration, rectal administration, intraperitoneal injection, intravascular injection, subcutaneous injection, transcutaneous administration, and intramuscular injection. The mammal includes, but is not limited to a mammal diagnosed as having one or more symptoms of atherosclerosis or diagnosed as at risk for stroke or atherosclerosis. The mammal includes, but is not limited to a mammal diagnosed as having one or more symptoms of atherosclerosis or diagnosed as at risk for stroke or atherosclerosis.

In still another embodiment, this invention provides a method of reducing or inhibiting one or more symptoms of osteoporosis in a mammal. The method typically involves administering to the mammal one or more small organic molecule(s) described herein, where the small organic molecule is administered in a concentration sufficient to reduce or eliminate one or more symptoms of osteoporosis. In certain embodiments, the small organic molecule(s) are administered in a concentration sufficient to reduce or eliminate decalcification of a bone. In certain embodiments, the small organic molecule(s) are administered in a concentration sufficient to induce recalcification of a bone. The small organic molecule(s) can be combined with a pharmacologically acceptable excipient (e.g., an excipient suitable for oral administration to a mammal).

Definitions.

The term “ameliorating” when used with respect to “ameliorating one or more symptoms of atherosclerosis” refers to a reduction, prevention, or elimination of one or more symptoms characteristic of atherosclerosis and/or associated pathologies. Such a reduction includes, but is not limited to a reduction or elimination of oxidized phospholipids, a reduction in atherosclerotic plaque formation and rupture, a reduction in clinical events such as heart attack, angina, or stroke, a decrease in hypertension, a decrease in inflammatory protein biosynthesis, reduction in plasma cholesterol, and the like. “Ameliorating one or more symptoms of atherosclerosis” can also refer to improving blood flow to vascular beds affected by atherosclerosis.

The term “protecting group” or “blocking group” refers to a chemical group that, when attached to a functional group in an amino acid (e.g. a side chain, an alpha amino group, an alpha carboxyl group, etc.) blocks or masks the properties of that functional group. Preferred amino-terminal protecting groups include, but are not limited to acetyl, or amino groups. Other amino-terminal protecting groups include, but are not limited to alkyl chains as in fatty acids, propionyl, formyl and others. Preferred carboxyl terminal protecting groups include, but are not limited to groups that form amides or esters. The term “side chain protection groups” refers to protecting groups that protect/block a reactive group on a molecule (e.g., an R group of an amino acid, an amino or carboxyl group, e.g., of an amino acid, etc.). Protecting groups include, but are not limited to amino protecting groups, carboxylprotecting groups and hydroxylprotecting groups such as aryl ethers and guanidine protecting groups such as nitro, tosyl etc.

The phrase “protect a phospholipid from oxidation by an oxidizing agent” refers to the ability of a compound to reduce the rate of oxidation of a phospholipid (or the amount of oxidized phospholipid produced) when that phospholipid is contacted with an oxidizing agent (e.g. hydrogen peroxide, 13-(S)-HPODE, 15-(S)-HPETE, HPODE, HPETE, HODE, HETE, etc.).

The terms “low density lipoprotein” or “LDL” is defined in accordance with common usage of those of skill in the art. Generally, LDL refers to the lipid-protein complex which when isolated by ultracentrifugation is found in the density range d=1.019 to d=1.063.

The terms “high density lipoprotein” or “HDL” is defined in accordance with common usage of those of skill in the art. Generally “HDL” refers to a lipid-protein complex which when isolated by ultracentrifugation is found in the density range of d=1.063 to d=1.21.

The term “Group I HDL” refers to a high density lipoprotein or components thereof (e.g. apo A-I, paraoxonase, platelet activating factor acetylhydrolase, etc.) that reduce oxidized lipids (e.g. in low density lipoproteins) or that protect oxidized lipids from oxidation by oxidizing agents.

The term “Group II HDL” refers to an HDL that offers reduced activity or no activity in protecting lipids from oxidation or in repairing (e.g. reducing) oxidized lipids.

The term “HDL component” refers to a component (e.g. molecules) that comprises a high density lipoprotein (HDL). Assays for HDL that protect lipids from oxidation or that repair (e.g. reduce oxidized lipids) also include assays for components of HDL (e.g. apo A-I, paraoxonase, platelet activating factor acetylhydrolase, etc.) that display such activity.

A “monocytic reaction” as used herein refers to monocyte activity characteristic of the “inflammatory response” associated with atherosclerotic plaque formation. The monocytic reaction is characterized by monocyte adhesion to cells of the vascular wall (e.g. cells of the vascular endothelium), and/or chemotaxis into the subendothelial space, or generation of monocyte chemotactic activity, and/or differentiation of monocytes into macrophages.

The term “absence of change” when referring to the amount of oxidized phospholipid refers to the lack of a detectable change, more preferably the lack of a statistically significant change (e.g. at least at the 85%, preferably at least at the 90%, more preferably at least at the 95%, and most preferably at least at the 98% or 99% confidence level). The absence of a detectable change can also refer to assays in which oxidized phospholipid level changes, but not as much as in the absence of the protein(s) described herein or with reference to other positive or negative controls.

The following abbreviations are used herein: PAPC: L-α-1-palmitoyl-2-arachidonoyl-sn-glycero-3-phosphocholine; POVPC: 1-palmitoyl-2-(5-oxovaleryl)-sn-glycero-3-phosphocholine; PGPC: 1-palmitoyl-2-glutaryl-sn-glycero-3-phosphocholine; PEIPC: 1-palmitoyl-2-(5,6-epoxyisoprostane E₂)-sn-glycero-3-phsophocholine; ChC18:2: cholesteryl linoleate; ChC18:2-OOH: cholesteryl linoleate hydroperoxide; DMPC: 1,2-ditetradecanoyl-rac-glycerol-3-phosphocholine; PON: paraoxonase; HPF: Standardized high power field; PON: paraoxonase; BL/6: C57BL/6J; C3H:C3H/HeJ.

The terms “coadministering” or “concurrent administration”, when used, for example with respect to a small organic molecule of this invention and another active agent (e.g., a statin), refers to administration of the small organic molecule and the active agent such that both can simultaneously achieve a physiological effect. The two agents, however, need not be administered together. In certain embodiments, administration of one agent can precede administration of the other, however, such coadministering typically results in both agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

The term “detoxify” when used with respect to lipids, LDL, or HDL refers the removal of some or all oxidizing lipids and/or oxidized lipids. Thus, for example, the uptake of all or some HPODE and/or HPETE (both hydroperoxides on fatty acids) will prevent or reduce entrance of these peroxides into LDLs and thus prevent or reduce LDL oxidation.

The term “pre-beta high density lipoprotein-like particles” typically refers to cholesterol containing particles that also contain apoA-I and which are smaller and relatively lipid-poor compared to the lipid: protein ratio in the majority of HDL particles. When plasma is separated by FPLC, these “pre-beta high density lipoprotein-like particles” are found in the FPLC fractions containing particles smaller than those in the main HDL peak and are located to the right of HDL in an FPLC chromatogram as shown in related application U.S. Ser. No. 10/423,830.

The phrase “reverse lipid transport and detoxification” refers to the removal of lipids including cholesterol, other sterols including oxidized sterols, phospholipids, oxidizing agents, and oxidized phospholipids from tissues such as arteries and transport out of these peripheral tissues to organs where they can be detoxified and excreted such as excretion by the liver into bile and excretion by the kidneys into urine. Detoxification also refers to preventing the formation and/or destroying oxidized phospholipids as explained herein.

The term “biological sample” as used herein refers to any sample obtained from a living organism or from an organism that has died. Examples of biological samples include body fluids, tissue specimens, cells and cell lines taken from an organism (e.g. a human or non-human mammal).

The term “amide” when referring to a hydrophobic protecting group or a hydrophobic blocking group includes a simple amide to methylamide or ethylamide. The term also includes alkyl amides such as CO—NH—R where R is methyl, ethyl, etc. (e.g. up to 7, preferably 9, more preferably 11 or 13 carbons).

The term “an amino acid R group” refers to a a chemical group that can be found on the alpha carbon of the amino acid that typically does not participate in peptide bond formation when the amino acid is present in a protein and that typically determines the “species” of amino acid. The phrase “an R group from an amino acid” or an “amino acid R group” indicates that the chemical group in question can be found in a natural or non-natural amino acid. In the context of the present inenvention that R group need not be derived from an amino acid (e.g., the R group can be synthesized de novo, derived by reaction with another chemical species, etc.). A list of illustrative R groups is provided in Table 1. This list is intended to be illustrative and not limiting.

TABLE 1
Illustrative amino acid R groups.

| Amino Acid | R Group |
|---|---|
| Alanine | —CH₃ |
| Arginine | 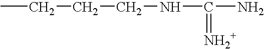 |
| Asparagine | 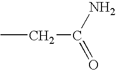 |
| Aspartic Acid | —CH₂—COO⁻ |
| Cysteine | —CH₂—SH |
| Glutamic Acid | —CH₂—CH₂—COO⁻ |
| Glutamine | 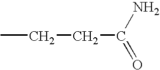 |
| Glycine | —H |
| Histidine | 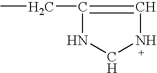 |
| Isoleucine | 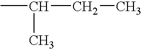 |
| Leucine | 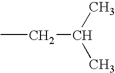 |
| Lysine | —CH₂—CH₂—CH₂—CH₂—NH₃ |
| Methionine | —CH₂—CH₂—S—CH₃ |
| Norleucine | —CH₂—CH₂—CH₂—CH₃ |
| Phenylalanine | 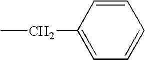 |
| Proline | 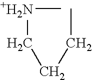 |
| Serine | —CH₂—OH |
| Threonine | 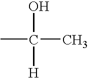 |
| Tryptophan | 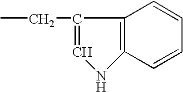 |
| Tyrosine | 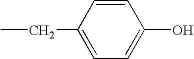 |
| Valine | 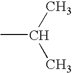 |

A molecule or composition that “converts pro-inflammatory HDL to anti-inflammatory HDL or makes anti-inflammatory HDL more anti-inflammatory” refers to a molecule or composition that when administered to a mammal (e.g. a human, a rat, a mouse, etc.), or that when used in an appropriate ex vivo assay (e.g. as described herein), converts HDL to an HDL that reduces or blocks lipid oxidation by an oxidizing agent (e.g. as described in U.S. Ser. No. 6,596,544), and/or that has increased paraoxonase activity, and/or that decreases LDL-induced monocyte chemotactic activity generated by artery wall cells as compared to HDL in a control assay (e.g. HDL from a control animal or assay administered a lower dose of the molecule or a negative control animal or assay lacking the molecule). The alteration of HDL (conversion from non-protective to protective or increase in protective activity) is preferably a detectable change. In preferred embodiments, the change is a statistically significant change, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level. In certain embodiments, the change is at least a 10% change, preferably at least a 20% change, more preferably at least a 50% change and most preferably at least a 90% change.

### BRIEF DESCRIPTION OF THE DRAWINGS

FIG. 1A shows a peptide that mitigates a symptom of atherosclerosis (Boc-Lys(εBoc)-Arg-Glu-Ser(tBu)-OtBu, SEQ ID NO:4 [SEQ ID NO:258 in copending U.S. Ser. No. 10/649,378, filed on Aug. 26, 2003]), while FIG. 1B shows to a non-peptide analogue of the present invention (2-(t-butyl hydroxymethyl)-5-carboxyethyl-8-guanidinopropyl-11-phenylbutyl-12-phenyl-dodecanoic acid t-butyl ester).

FIGS. 2A and 2B illustrate one synthesis scheme for a molecule of this invention.

FIG. 3 shows the solubility of peptides in ethyl acetate. SEQ ID NO:3: Boc-Lys(εBoc)-Glu-Arg-Ser(tBu)-OtBu; and SEQ ID NO:4: Boc-Lys(εBoc)-Arg-Glu-Ser(tBu)-OtBu. Also shown is the solubility in ethyl acetate of SEQ ID NO:5 (SEQ ID NO:250 of U.S. Ser. No. 10/649,378).

FIG. 4 SEQ ID NO:4 forms 7.5 nm particles when mixed with DMPC in an aqueous environment. To 1 mg/ml of DMPC suspension in phosphate buffered saline (PBS) was added 10% deoxycholate until the DMPC was dissolved. SEQ ID NO: 4 or SEQ ID NO:3 were added (DMPC: peptide; 1:10; wt:wt) and the reaction mixture dialyzed. After dialysis the solution remained clear with SEQ ID NO:4 but was turbid after the deoxycholate was removed by dialysis in the case of SEQ ID NO:3. The figure is an electron micrograph prepared with negative staining and at 147,420× magnification. The arrows indicate SEQ ID NO: 4 particles measuring 7.5 nm (they appear as small white particles).

FIG. 5 the peptide of SEQ ID NO:4 added to DMPC in an aqueous environment forms particles with a diameter of approximately 7.5 nm (large open), and stacked lipid-peptide bilayers (large striped arrow) (small arrows pointing to the white lines in the cylindrical stack of disks) with a bilayer dimension on the order of 3.4 to 4.1 nm with spacing between the bilayers (black lines between white lines in the stack of disks) of approximately 2 nm. The conditions and magnifications are the same as described in FIG. 4.

FIG. 6 shows that the peptide of SEQ ID NO:4 added to DMPC in an aqueous environment forms stacked lipid-peptide bilayers (striped arrow) and vesicular structures of approximately 38 nm white arrows).

FIG. 7 shows that DMPC in an aqueous environment without SEQ ID NO:4 does not form particles with a diameter of approximately 7.5 nm, or stacked lipid-peptide bilayers, nor vesicular structures of approximately 38 nm. The DMPC vesicles shown are 12.5-14 nm. The conditions and magnifications are the same as described in FIG. 4.

FIG. 8 shows a molecular model of the peptide of SEQ ID NO:3 compared to the peptide of SEQ ID NO:4. Red represents oxygen, blue represents nitrogen, gray represents carbon, and white represents hydrogen molecules.

FIG. 9 shows a space-filling molecule model of SEQ ID NO:3 compared to SEQ ID NO:4. The arrows in this space filling molecular model identify the polar and non-polar portions of the molecules. The color code is the same as in FIG. 8.

FIG. 10 illustrates peptide backbones (in the bottom panels) for the orientations given in the top panels.

FIG. 11 shows molecular models of SEQ ID NO:3 compared to SEQ ID NO:4 identifying the Ser(tBu)-OtBu groups. The color code is as in FIG. 8.

FIG. 12 shows molecular models of SEQ ID NO:3 compared to SEQ ID NO:4 identifying various blocking groups. The color code is as in FIG. 8.

FIG. 13 shows that SEQ ID NO:4 (but not SEQ ID NO:3) renders apoE null HDL anti-inflammatory.

FIG. 14 shows that the peptide of SEQ ID NO:4, but not the peptide of SEQ ID NO:3, significantly decreases aortic root atherosclerosis in apoE null mice. The aortic root (aortic sinus) lesion score was determined in the apoE null mice described in FIG. 13. The number of mice in each group is shown (n=) at the bottom of the figure and a representative section for each group is shown at the top of the figure.

FIG. 15 shows that the peptide of SEQ ID NO:4 but not SEQ ID NO:3 significantly decreases aortic atherosclerosis in en face preparations in apoE null mice. The percent aortic surface containing atherosclerotic lesions was determined in en face preparations in the apoE null mice described in FIG. 13. The number of mice in each group is shown (n=) at the bottom of the left panel and a representative aorta for mice fed chow alone or chow supplemented with. SEQ ID NO:4 is shown in the right panel.

FIG. 16 shows that SEQ ID NO:5 (SEQ ID NO:250 from U.S. Ser. No. 10/649,378 synthesized from all L-amino acids significantly decreases atherosclerosis. ApoE null mice (20 per group) were maintained on a chow diet (Chow) or on chow supplemented with 200 μg/gm chow of SEQ ID NO:5 (250) synthesized from all L-amino acids. After 12 weeks the mice were sacrificed and the % Aortic Surface Area with Lesions was determined in en face preparations. *p=0.012.

### DETAILED DESCRIPTION

This invention pertains to the discovery of a class of small organic molecules that are able to associate with phospholipids and exhibit certain biological properties similar to human apo-A-I. In particular, it was a discovery that these small organic molecule stimulate the formation and cycling of pre-beta high density lipoprotein-like particles. In addition, these molecules are capable of enhancing/synergizing the effect of statins allowing statins to be administered as significantly lower dosages or to be significantly more anti-inflammatory at any given dose. It was also discovered that the molecules described herein can inhibit and/or prevent and/or treat one or more symptoms of atherosclerosis, osteoporosis, diabetes, and the like. The molecules described herein can also increase pre-beta HDL; and/or increase HDL paraoxonase activity.

Moreover, molecules described herein are believed to be effective for oral delivery, show elevated serum half-life, and the ability to mitigate or prevent/inhibit one or more symptoms of atherosclerosis.

It a surprising discovery that the small organic molecules of this invention also possess significant anti-inflammatory properties. Without being bound to a particular theory, it is believed that the small organic molecules bind the “seeding molecules” required for the formation of pro-inflammatory oxidized phospholipids such as Ox-PAPC, POVPC, PGPC, and PEIPC. Since many inflammatory conditions are mediated at least in part by oxidized lipids, we believe the molecules of this invention are effective in ameliorating conditions that are known or suspected to be due to the formation of biologically active oxidized lipids. These include, but are not limited to atherosclerosis, rheumatoid arthritis, lupus erythematous, polyarteritis nodosa, and osteoporosis.

I. Small Organic Molecules.

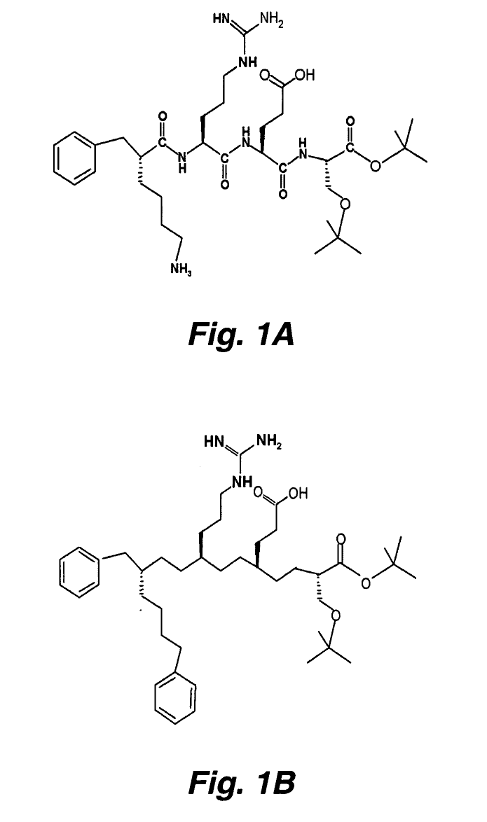

In certain embodiments, the small organic molecules are similar to, and in certain cases, mimetics of the tetra- and penta-peptides described in copending application U.S. Ser. No. 10/649,378, filed on Aug. 26, 2003 and U.S. Ser. No. 60/494,449, filed on Aug. 11, 2003, which are incorporated herein by reference. Thus, for example, FIG. 1A shows a small peptide (Boc-Lys(εBoc)-Arg-Glu-Ser(tBu)-OtBu, (SEQ ID NO:2), while FIG. 1B shows 2-(t-butyl hydroxymethyl)-5-carboxyethyl-8-guanidinopropyl-11-phenylbutyl-12-phenyl-dodecanoic acid t-butyl ester, a nonpeptide analog that is a small organic molecule in accordance with the present invention.

The small organic molecules of this invention typically have molecular weights less than about 900 Daltons. Typically the molecules are are highly soluble in ethyl acetate (e.g., at concentrations equal to or greater than 4 mg/mL), and also are soluble in aqueous buffer at pH 7.0.

Contacting phospholipids such as 1,2-dimyristoyl-sn-glycero-3-phosphocholine (DMPC), with molecules of this invention in an aqueous environment typically results in the formation of particles with a diameter of approximately 7.5 nm (+0.1 nm). In addition, stacked bilayers are often formed with a bilayer dimension on the order of 3.4 to 4.1 nm with spacing between the bilayers in the stack of approximately 2 nm. Vesicular structures of approximately 38 nm are also often formed. Moreover, when the molecules of this invention are administered to a mammal they render HDL more anti-inflammatory and mitigate one or more symptoms of atherosclerosis and/or other conditions characterized by an inflammatory response.

Thus, in certain embodiments, the small organic molecule is one that ameliorates one or more symptoms of a pathology characterized by an inflammatory response in a mammal (e.g. atherosclerosis), where the small molecule is soluble in in ethyl acetate at a concentration greater than 4 mg/mL, is soluble in aqueous buffer at pH 7.0, and, when contacted with a phospholipid in an aqueous environment, forms particles with a diameter of approximately 7.5 nm and forms stacked bilayers with a bilayer dimension on the order of 3.4 to 4.1 nm with spacing between the bilayers in the stack of approximately 2 nm, and has a molecular weight les than 900 daltons.

In certain embodiment, the molecule has the formula: 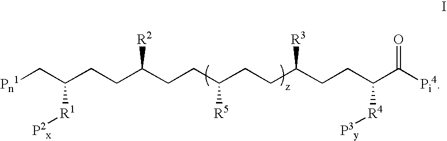
where P¹, P², P³, and P⁴ are independently selected hydrophobic protecting groups; R¹ and R⁴ are independently selected amino acid R groups; n, i, x, y, and z are independently zero or 1 such that when n and x are both zero, R² is a hydrophobic group and when y and i are both zero, R⁴ is a hydrophobic group; R² and R³ are acidic or basic groups at pH 7.0 such that when R² is acidic, R³ is basic and when R² is basic, R³ is acidic; and R⁵, when present is selected from the group consisting of an aromatic group, an aliphatic group, a postively charged group, or a negatively charged group. In certain embodiments, R² or R³ is —(CH₂)j-COOH where j=1, 2, 3, or 4 and/or —(CH₂)j-NH₂ where j=1, 2, 3, 4, or 5, or —(CH₂)j-NH—C(═NH)—NH₂ where n=1, 2, 3 or 4. In certain embodiments, R², R³, and R⁵, when present, are amino acid R groups. Thus, for example, In various embodiments R² and R³ are independently an aspartic acid R group, a glutamic acid R group, a lysine R group, a histidine R group, or an arginine R group (e.g., as illustrated in Table 1).

In certain embodiments, R¹ is selected from the group consisting of a Lys R group, a Trp R group, a Phe R group, a Leu R group, an Orn R group, pr a norLeu R group. In certain embodiments, R⁴ is selected from the group consisting of a Ser R group, a Thr R group, an Ile R group, a Leu R group, a norLeu R group, a Phe R group, or a Tyr R group.

In various embodiments x is 1, and R⁵ is an aromatic group (e.g., a Trp R group).

In various embodiments at least one of n, x, y, and i is 1 and P¹, P², P³, and P⁴ when present, are independently selected from the group consisting of polyethylene glycol (PEG), an acetyl, amide, a 3 to 20 carbon alkyl group, fmoc, 9-fluoreneacetyl group, 1-fluorenecarboxylic group, 9-fluorenecarboxylic, 9-fluorenone-1-carboxylic group, benzyloxycarbonyl, xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), benzyloxy (BzlO), benzyl (Bzl), benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), benzyloxymethyl (Bom), t-butoxycarbonyl (Boc), cyclohexyloxy (cHxO), t-butoxymethyl (Bum), t-butoxy (tBuO), t-Butyl (tBu), a propyl group, a butyl group, a pentyl group, a hexyl group, and trifluoroacetyl (TFA). In certain embodiments, P¹ when present and/or P² when present are independently selected from the group consisting of Boc-, Fmoc-, and Nicotinyl- and/or P³ when present and/or P⁴ when present are independently selected from the group consisting of tBu, and OtBu.

While a number of protecting groups (P¹, P², P³, P⁴) are illustrated above, this list is intended to be illustrative and not limiting. In view of the teachings provided herein, a number of other protecting/blocking groups will also be known to one of skill in the art. Such blocking groups can be selected to minimize digestion (e.g., for oral pharmaceutical delivery), and/or to increase uptake/bioavailability (e.g., through mucosal surfaces in nasal delivery, inhalation therapy, rectal administration), and/or to increase serum/plasma half-life. In certain embodiments, the protecting groups can be provided as an excipient or as a component of an excipient.

In certain embodiments, z is zero and the molecule has the formula: 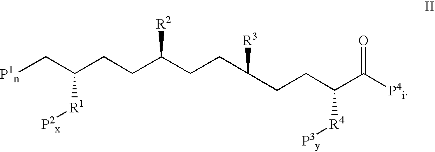
where P¹, P², P³, P⁴, R¹, R², R³, R⁴, n, x, y, and i are as described above.

In certain embodiments, z is zero and the molecule has the formula: 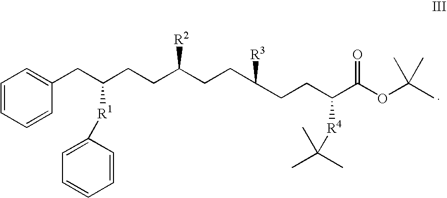
where R¹, R², R³, and R⁴ are as described above.

In one embodiment, the molecule has the formula:

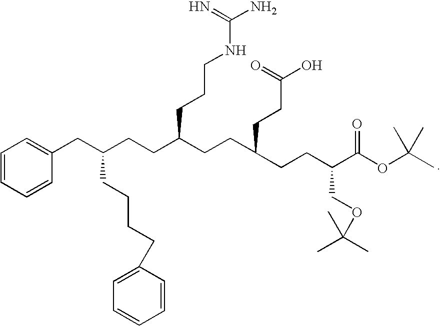

In certain embodiments, this invention contemplates small molecules having one or more of the physical and/or functional properties described herein and having the formula: 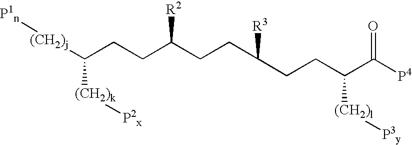
where P¹, P², P³, and P⁴ are independently selected hydrophobic protecting groups as described above, n, x, and y are independently zero or 1; j, k, and l are independently zero, 1, 2, 3, 4, or 5; and R² and R³ are acidic or basic groups at pH 7.0 such that when R² is acidic, R³ is basic and when R² is basic, R³ is acidic. In certain preferred embodiments, the small molecule is soluble in water; and the small molecule has a molecular weight less than about 900 Daltons. In certain embodiments, n, x, y, j, and l are 1; and k is 4.

In certain embodiments, P¹ and/or P² are aromatic protecting groups. In certain embodiments, R² and R³ are amino acid R groups, e.g., as described above. In various embodiments least one of n, x, and y, is 1 and P¹, P², P³ and P⁴ when present, are independently protecting groups, e.g. as described above.selected from the group consisting of polyethylene glycol (PEG), an acetyl, amide, 3 to 20 carbon alkyl groups, Fmoc, 9-fluoreneacetyl group, 1-fluorenecarboxylic group, 9-fluorenecarboxylic, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-penta.

In various embodiemtns, this invention expressly includes, but is not limited to enantiomers and/or mixtures of molecules of different chirality, of the various molecules illustrated in the formulas herein.

II. Synthesizing Small Organic Molecules of this Invention.

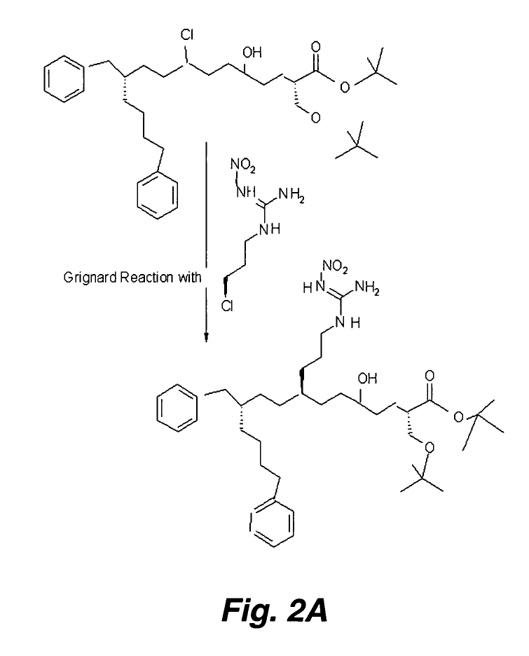

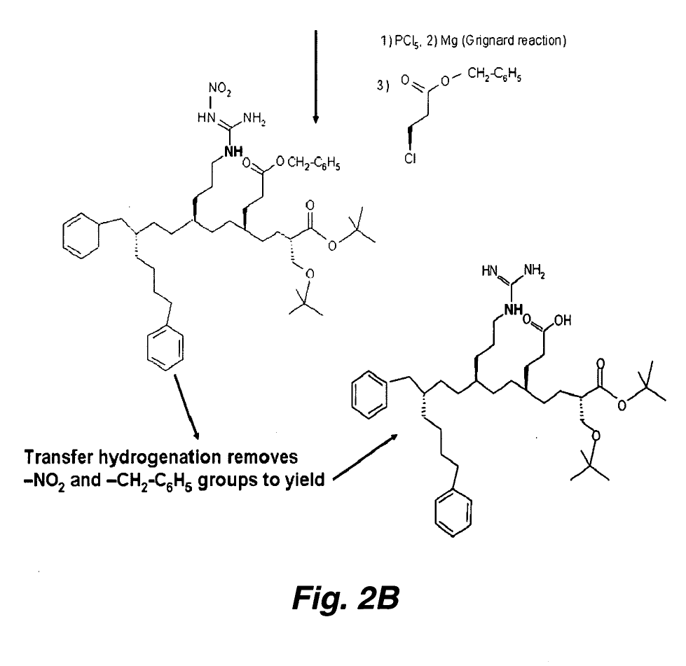

The molecules of this invention are relatively small (typically less than about 900 Daltons) and are readily synthesized using standard methods well known to those of skill in the art. FIGS. 2A and 2B illustrate a typical synthesis scheme for compounds of the present invention. While these figures specifically illustrate the synthesis of a compound of as shown in FIG. 1B, one of skill in the art that appropriate chain elongation, derivatization and Grignard reaction can be used to obtain any of the other molecules described herein (see, e.g., Calvin A. Buehler and Donald E. Pearson (1970) Survey of Organic Synthesis Wiley Interscience New York; Anantharamaiah and Roeske (1982) Tetrahedran Letter 23: 3335-3338).

III. Functional Assays of Small Molecules.

Certain molecules of this invention are desctribed herein by various formulas (e.g. Formula I or II, or III, above). In certain embodiments, however, preferred molecules of this invention are characterized by one or more of the following functional properties (e.g., in addition to the physical properties described above):
1 They convert pro-inflammatory HDL to anti-inflammatory HDL or make anti-inflammatory HDL more anti-inflammatory;
2 They decrease LDL-induced monocyte chemotactic activity generated by artery wall cells;
3 They stimulate the formation and cycling of pre-β HDL;
4 They raise HDL cholesterol; and/or
5 They increase HDL paraoxonase activity.

The molecules disclosed herein, and/or other molecules meeting the physical limitations described herein can readily be tested for one or more of these activities as desired.

Methods of screening for each of these functional properties are well known to those of skill in the art. In particular, it is noted that assays for monocyte chemotactic activity, HDL cholesterol, and HDL HDL paraoxonase activity are illustrated in PCT/US01/26497 (WO 02/15923). Assays for determining HDL inflammatory and/or anti-inflammatory properties can be performed as described below.

### A) Determination of HDL Inflammatory/Anti-inflammatory Properties—

1) Monocyte Chemotactic Activity (MCA) Assay

Lipoproteins, human artery wall cocultures, and monocytes can be prepared and monocyte chemotactic activity (MCA) determined as previously described (Van Lenten et al. (2002) Circulation, 106: 1127-1132). Induction of MCA by a standard control LDL can be determined in the absence or presence of the subject's HDL. Values in the absence of HDL are typically normalized to 1.0. Values greater than 1.0 after the addition of HDL indicate pro-inflammatory HDL; values less than 1.0 indicate anti-inflammatory HDL.

2) Cell-free Assay—

The cell-free assay was a modification of a previously published method using PEIPC as the fluorescence-inducing agent. In one embodiment, HDL is isolated by dextran sulfate method. Sigma “HDL cholesterol reagent” (Catalog No. 352-3) containing dextran sulfate and magnesium ions is dissolved in distilled water (10.0 mg/ml). Fifty microliters of dextran sulfate solution is mixed with 500 μl of the test plasma and incubated at room temperature for 5 min and subsequently centrifuged at 8,000 g for 10 min. The supernatant containing HDL is used in the experiments after cholesterol determination using a cholesterol assay kit (Cat. No. 2340-200, Thermo DMA Company, Arlington, Tex.).

We have previously reported (Navab et al. (2001) J Lipid Res, 1308-1317) that HDL isolated by this method inactivates bioactive phospholipids to a similar extent as compared with HDL that has been isolated by conventional ultracentrifuge methods. To determine the inflammatory/anti-inflammatory properties of HDL samples from patients and controls, the change in fluorescence intensity as a result of the oxidation of DCFH by PEIPC in the absence or presence of the test HDL can be used. Thus, for example, DCFH-DA is dissolved in fresh methanol at 2.0 mg/ml and incubated at room temperature and protected from light for 30 min. resulting in the release of DCFH. The assay can be adapted for analyzing a large number of samples with a plate reader. Flat-bottom, black, polystyrene microtiter plates (Microfluor2, Cat. No. 14-245-176, Fisher) can be utilized for this purpose. Ten μl of PEIPC solution (final concentration of 50 μg/ml), and 90 μl of HDL-containing dextran sulfate supernatant (final concentration of 10 μg/ml cholesterol), were aliquoted into microtiter plates and mixed. The plates were then incubated at 37° C. on a rotator for 1.0 hr. Ten μl of DCFH solution (0.2 mg/ml) is then added to each well, mixed and incubated for an additional 2 hrs at 37° C. with rotation. The fluorescence is subsequently determined with a plate reader (Spectra Max, Gemini XS; Molecular Devices) at an excitation wavelength of 485 nm and emission wavelength of 530 nm and cutoff of 515 nm with the photomultiplier sensitivity set at “medium”. Typical values for intra- and interassay variability have been 5.3±1.7% and 7.1±3.2%, respectively. Values in the absence of HDL are normalized to 1.0. Values greater than 1.0 after the addition of the test HDL indicate pro-inflammatory HDL; values less than 1.0 indicate anti-inflammatory HDL.

3) Other Procedures

Plasma levels of interleukin-6 (IL-6) and tumor necrosis factor-α (TNF-α) can be determined by previously published methods (Scheidt-Nave et al. (2001) J Clin Endocrinol Metab., 86:2032-2042; Piguet et al. (1987) J Experiment Med., 166, 1280-1289). Plasma total cholesterol, triglycerides, LDL-cholesterol, HDL-cholesterol and glucose can also be determined as previously described (Navab et al. (1997) J Clin Invest, 99:2005-2019) using kits (Sigma), and hs-CRP levels (Rifai et al. (1999) Clin Chem., 45:2136-2141) can be determined using a sandwich enzyme immunoassay from Immunodiagnostik (ALPCO Diagnostics, Windham, N.H.). Statistical significance is determined, e.g., with model I ANOVA, and significance can be defined as a value of p<0.05.

It is noted that these methods are merely illustrative and not intended to be limiting. Using the teachings provided herein, other assays for the desired functional properties of the molecules of this invention can readily be provided.

IV. Stimulating the Formation and Cycling of Pre-Beta High Density Lipoprotein-Like Particles.

Reverse cholesterol transport is considered to be important in preventing the build up of lipids that predisposes to atherosclerosis (Shah et al. (2001) Circulation, 103: 3047-3050.) Many have believed the lipid of consequence is cholesterol. Our laboratory has shown that the key lipids are oxidized phospholipids that initiate the inflammatory response in atherosclerosis (Navab et al. (2001) Arterioscler Thromb Vasc Biol., 21(4): 481-488; Van Lenten et al. (001) Trends Cardiovasc Med, 11: 155-161; Navab M et al. (2001) Circulation, 104: 2386-2387).

This inflammatory response is also likely responsible for plaque erosion or rupture that leads to heart attack and stroke. HDL-cholesterol levels are inversely correlated with risk for heart attack and stroke (Downs et al. (1998) JAMA 279: 1615-1622; Gordon et al. (1977) Am J. Med., 62: 707-714; Castelli et al. (1986) JAMA, 256: 2835-2838).

Pre-beta HDL is generally considered to be the most active HDL fraction in promoting reverse cholesterol transport (e.g., picking up cholesterol from peripheral tissues such as arteries and carrying it to the liver for excretion into the bile; see, Fielding and Fielding (2001) Biochim Biophys Acta, 1533(3): 175-189). However, levels of pre-beta HDL can be increased because of a failure of the pre-beta HDL to be cycled into mature alpha-migrating HDL e.g. LCAT deficiency or inhibition (O'Connor et al. (1998) J Lipid Res, 39: 670-678). High levels of pre-beta HDL have been reported in coronary artery disease patients (Miida et al. (1996) Clin Chem., 42: 1992-1995).

Moreover, men have been found to have higher levels of pre-beta HDL than women but the risk of men for coronary heart disease is greater than for women (O'Connor et al. (1998) J Lipid Res., 39: 670-678). Thus, static measurements of pre-beta HDL levels themselves are not necessarily predictive of risk for coronary artery disease. The cycling, however, of cholesterol through pre-beta HDL into mature HDL is universally considered to be protective against atherosclerosis (Fielding and Fielding (2001) Biochim Biophys Acta, 1533(3): 175-189). Moreover, we have demonstrated that the removal of oxidized lipids from artery wall cells through this pathway protects against LDL oxidation.

Without being bound to a particular theory, it is believed that after administration of the molecules of this ivnetion, the molecules will participate in the formation of small pre-beta HDL-like particles that contain relatively high amounts of apoA-I and paraoxonase. It is believed that the molecules act as a catalyst causing the formation of these pre-beta HDL-like particles. The molecules of this invention are believed to recruit amounts of apoA-I, paraoxonase, and cholesterol into these particles that are orders of magnitude more than the amount of small organic molecule itself.

Thus, following absorption, it is believed the small molecules of this invention, rapidly recruit relatively large amounts of apoA-I and paraoxonase to form pre-beta HDL-like particles which are very likely the most potent particles for both promoting reverse cholesterol transport and for destroying biologically active oxidized lipids. We believe that the formation of these particles and their subsequent rapid incorporation into mature HDL results in dramatic reduction in atherosclerotic symptoms.

Thus, in one embodiment, this invention provides methods of stimulating the formation and cycling of pre-beta high density lipoprotein-like particles by administration of one or more small organic molecules as described herein. The molecules can thereby promote lipid transport and detoxification. In various embodiments, the molecule(s) can be administered in conjunction with one or more of the peptides described in U.S. Pat. No. 6,664,230, and/or in PCT Publications WO 02/15923, WO 2004/034977, PCT/US2004/026288, PCT/US03/09988, and the like.

V. Mitigation of a Symptom of Atherosclerosis.

We discovered that normal HDL inhibits three steps in the formation of mildly oxidized LDL. In those studies (see, e.g., U.S. Pat. No. 6,664,230, and PCT Applications WO 02/15923 and WO 2004/034977) we demonstrated that treating human LDL in vitro with apo A-I or an apo A-I mimetic peptide (37 pA) removed seeding molecules from the LDL that included HPODE and HPETE. These seeding molecules were required for cocultures of human artery wall cells to be able to oxidize LDL and for the LDL to induce the artery wall cells to produce monocyte chemotactic activity. We also demonstrated that after injection of apo A-I into mice or infusion into humans, the LDL isolated from the mice or human volunteers after injection/infusion of apo A-I was resistant to oxidation by human artery wall cells and did not induce monocyte chemotactic activity in the artery wall cell cocultures.

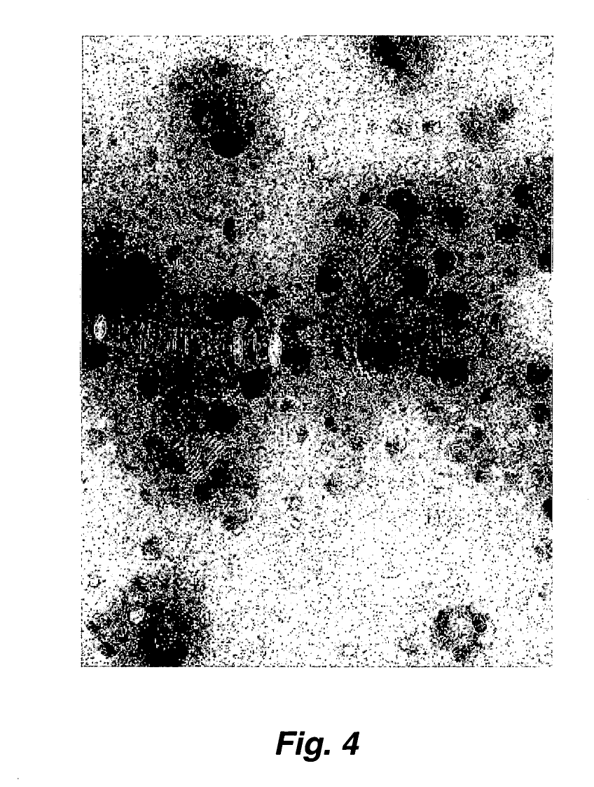

The protective function of certain peptides of which the molecules of this invention are mimetics/analogues is illustrated, for example, U.S. Pat. No. 6,664,230, and PCT Applications WO 02/15923 and WO 2004/034977, see, e.g., FIGS. 1-5 in WO 02/15923. FIG. 1, panels A, B, C, and D in WO 02/15923 show the association of ¹⁴C-D-5F with blood components in an ApoE null mouse. It is also demonstrated that HDL from mice that were fed an atherogenic diet and injected with PBS failed to inhibit the oxidation of human LDL and failed to inhibit LDL-induced monocyte chemotactic activity in human artery wall coculures. In contrast, HDL from mice fed an atherogenic diet and injected daily with peptides described herein was as effective in inhibiting human LDL oxidation and preventing LDL-induced monocyte chemotactic activity in the cocultures as was normal human HDL (FIGS. 2A and 2B in WO 02/15923). In addition, LDL taken from mice fed the atherogenic diet and injected daily with PBS was more readily oxidized and more readily induced monocyte chemotactic activity than LDL taken from mice fed the same diet but injected with 20 μg daily of peptide 5F. The D peptide did not appear to be immunogenic (FIG. 4 in WO 02/15923).

In the assays performed to date, the molecules of the present invention show activity similar to that shown by the peptides discussed above. It is therefore believed that the small molecules of this invention can prevent progression of atherosclerotic lesions in mice fed an atherogenic diet.

Thus, in one embodiment, this invention provides methods for ameliorating and/or preventing one or more symptoms of atherosclerosis by administrating one or more of the small molecules described herein optionally in conjunction with one or more of the peptides described above. The molecules can be administered as a therapeutic, e.g., where one or more symptoms of atherosclerosis already exists or as a prophylactic to prevent the onset of atherosclerosis or symptoms thereof.

VI. Mitigation of a Symptom of Atheroscloerosis Associated with an Acute Inflammatory Response.

The molecules of this invention are also useful in a number of other contexts. For example, we have observed that cardiovascular complications (e.g., atherosclerosis, stroke, etc.) frequently accompany or follow the onset of an acute phase inflammatory response. Such an acute phase inflammatory response is often associated with a recurrent inflammatory disease (e.g., leprosy, tuberculosis, systemic lupus erythematosus, and rheumatoid arthritis), a viral infection (e.g., influenza), a bacterial infection, a fungal infection, an organ transplant, a wound or other trauma, an implanted prosthesis, a biofilm, and the like.

It is believed that administration of one or more of the small molecules described herein, can reduce or prevent the formation of oxidized phospholipids during or following an acute phase response and thereby mitigate or eliminate cardiovascular complications associated with such a condition.

Thus, for example, we have demonstrated that a consequence of influenza infection is the diminution in paraoxonase and platelet activating acetylhydrolase activity in the HDL. Without being bound by a particular theory, we believe that, as a result of the loss of these HDL enzymatic activities and also as a result of the association of pro-oxidant proteins with HDL during the acute phase response, HDL is no longer able to prevent LDL oxidation and was no longer able to prevent the LDL-induced production of monocyte chemotactic activity by endothelial cells.

We observed that in an animal subject injected with very low dosages of polyeptpides related to the molecules of this invention (see, e.g., U.S. Ser. No. 10/649,378, filed on Aug. 26, 2003 and U.S. Ser. No. 60/494,449, filed on Aug. 11, 2003) (e.g. 20 micrograms for mice) daily after infection with the influenza A virus paraoxonase levels did not fall and the biologically active oxidized phospholipids were not generated beyond background. This indicates that D-4F (and/or the small molecules of the present invention) can be administered to patients with known coronary artery disease during influenza infection or other events that can generate an acute phase inflammatory response (e.g., due to viral infection, bacterial infection, trauma, transplant, various autoimmune conditions, etc.) and thus we can prevent by this short term treatment the increased incidence of heart attack and stroke associated with pathologies that generate such inflammatory states.

Thus, in certain embodiments, this invention contemplates administering one or more of the molecules of this invention to a subject at risk for, or incurring, an acute inflammatory response and/or at risk for or incurring a symptom of atherosclerosis.

Thus, for example, a person having or at risk for coronary disease may prophylactically be administered a small molecule of this invention during flu season. A person (or animal) subject to a recurrent inflammatory condition, e.g., rheumatoid arthritis, various autoimmune diseases, etc., can be treated with a small molecule of this invention to mitigate or prevent the development of atherosclerosis or stroke. A person (or animal) subject to trauma, e.g. acute injury, tissue transplant, etc. can be treated with a small molecule of this invention to mitigate the development of atherosclerosis or stroke.

In certain instances such methods will entail a diagnosis of the occurrence or risk of an acute inflammatory response. The acute inflammatory response typically involves alterations in metabolism and gene regulation in the liver. It is a dynamic homeostatic process that involves all of the major systems of the body, in addition to the immune, cardiovascular and central nervous system. Normally, the acute phase response lasts only a few days; however, in cases of chronic or recurring inflammation, an aberrant continuation of some aspects of the acute phase response may contribute to the underlying tissue damage that accompanies the disease, and may also lead to further complications, for example cardiovascular diseases or protein deposition diseases such as amyloidosis.

One important aspect of the acute phase response is the radically altered biosynthetic profile of the liver. Under normal circumstances, the liver synthesizes a characteristic range of plasma proteins at steady state concentrations. Many of these proteins have important functions and higher plasma levels of these acute phase reactants (APRs) or acute phase proteins (APPs) are required during the acute phase response following an inflammatory stimulus. Although most APRs are synthesized by hepatocytes, some are produced by other cell types, including monocytes, endothelial cells, fibroblasts and adipocytes. Most APRs are induced between 50% and several-fold over normal levels. In contrast, the major APRs can increase to 1000-fold over normal levels. This group includes serum amyloid A (SAA) and either C-reactive protein (CRP) in humans or its homologue in mice, serum amyloid P component (SAP). So-called negative APRs are decreased in plasma concentration during the acute phase response to allow an increase in the capacity of the liver to synthesize the induced APRs.

In certain embodiments, the acute phase response, or risk therefore is evaluated by measuring one or more APPs. Measuring such markers is well known to those of skill in the art, and commercial companies exist that provide such measurement (e.g., AGP measured by Cardiotech Services, Louisville, Ky.).

VII. Synergizing the Activity of Statins.

It is also believed that the molecules of this invention have a synergistic effect when administered in conjunction with one or more statins. Thus, doses of the small organic molecule(s) alone, or statins alone, which by themselves have no effect on HDL function when given together will act synergistically.

Thus, in certain embodiments this invention provides methods for enhancing the activity of statins. The methods generally involve administering one or more molecules described herein concurrently (in conjunction with) one or more statins. The molecules described herein achieve synergistic action between the statin and the small organic molecule(s) to ameliorate atherosclerosis. In this context statins can be administered at significantly lower dosages thereby avoiding various harmful side effects (e.e., muscle wasting) associated with high dosage statin use and/or the anti-inflammatory properties of statins at any given dose are significantly enhanced.

VIII. Mitigation of a Symptom or Condition Associated with Coronary Calcification and/or Osteoporosis.

Vascular calcification and osteoporosis often co-exist in the same subjects (Ouchi et al. (1993) Ann NY Acad. Sci., 676: 297-307; Boukhris and Becker (1972) JAMA, 219: 1307-1311; Banks et al. (1994) Eur J Clin Invest., 24: 813-817; Laroche et al. (1994) Clin Rheumatol., 13: 611-614; Broulik and Kapitola (1993) Endocr Regul., 27: 57-60; Frye et al. (1992) Bone Mine., 19: 185-194; Barengolts et al. (1998) Calcif Tissue Int., 62: 209-213; Burnett and Vasikaran (2002) Ann Clin Biochem., 39: 203-210. Parhami et al. (Parhami et al. (1997) Arterioscl Thromb Vasc Biol., 17: 680-687) demonstrated that mildly oxidized LDL (MM-LDL) and the biologically active lipids in MM-LDL [i.e. oxidized 1-palmitoyl-2-arachidonoyl-sn-glycero-3-phosphorylcholine) (Ox-PAPC)], as well as the isoprostane, 8-iso prostaglandin E₂, but not the unoxidized phospholipid (PAPC) or isoprostane 8-iso progstaglandin F_{2α} induced alkaline phosphatase activity and osteoblastic differentiation of calcifying vascular cells (CVCs) in vitro, but inhibited the differentiation of MC3T3-E1 bone cells.

The osteon resembles the artery wall in that the osteon is centered on an endothelial cell-lined lumen surrounded by a subendothelial space containing matrix and fibroblast-like cells, which is in turn surrounded by preosteoblasts and osteoblasts occupying a position analogous to smooth muscle cells in the artery wall (Id.). Trabecular bone osteoblasts also interface with bone marrow subendothelial spaces (Id.). Parhami et al. postulated that lipoproteins could cross the endothelium of bone arteries and be deposited in the subendothelial space where they could undergo oxidation as in coronary arteries (Id.). Based on their in vitro data they predicted that LDL oxidation in the subendothelial space of bone arteries and in bone marrow would lead to reduced osteoblastic differentiation and mineralization which would contribute to osteoporosis (Id.). Their hypothesis further predicted that LDL levels would be positively correlated with osteoporosis as they are with coronary calcification (Pohle et al. (2001) Circulation, 104: 1927-1932) but HDL levels would be negatively correlated with osteoporosis (Parhami et al. (1997) Arterioscl Thromb Vasc Biol., 17: 680-687).

In vitro, the osteoblastic differentiation of the marrow stromal cell line M2-10B4 was inhibited by MM-LDL but not native LDL (Parhami et al. (1999) J Bone Miner Res., 14: 2067-2078). When marrow stromal cells from atherosclerosis susceptible C57BL/6 (BL6) mice fed a low fat chow diet were cultured there was robust osteogenic differentiation (Id.). In contrast, when the marrow stromal cells taken from the mice after a high fat, atherogenic diet were cultured they did not undergo osteogenic differentiation (Id.). This observation is particularly important since it provides a possible explanation for the decreased osteogenic potential of marrow stromal cells in the development of osteoporosis (Nuttall and Gimble (2000) Bone, 27: 177-184). In vivo the decrease in osteogenic potential is accompanied by an increase in adipogenesis in osteoporotic bone (Id.).

It is believed that administering one or more molecules of this invention alone, or in combination with one or more peptides described in U.S. Ser. No. 10/649,378, filed on Aug. 26, 2003 and U.S. Ser. No. 60/494,449, filed on Aug. 11, 2003, to apoE null mice will dramatically increase trabecular bone mineral density.

Our data indicate that osteoporosis can be regarded as an “atherosclerosis of bone”. It appears to be a result of the action of oxidized lipids. HDL destroys these oxidized lipids and promotes osteoblastic differentiation. This indicates that the small molecules described herein are useful for mitigation one or more symptoms of osteoporosis (e.g., for inhibiting decalcification) or for inducing recalcification of osteoporotic bone. The molecules are also useful as prophylactics to prevent the onset of symptom(s) of osteoporosis in a mammal (e.g. a patient at risk for osteoporosis).

Because of their efficacy in mitigating symptoms associated with an inflammatory response, and/or atherosclerosis, and/or osteoporosis (or other process associated with calcification ro decalcification), in certain embodiments, this invention contemplates the use of the molecules described herein to mitigate and/or to inhibit or prevent a symptom of a disease such as polymyalgia rheumatica, polyarteritis nodosa, scleroderma, lupus erythematosus, idiopathic pulmonary fibrosis, chronic obstructive pulmonary disease (e.g., asthma), Alzheimers Disease, AIDS, coronary calcification, calcific aortic stenosis, osteoporosis, and the like.

IX. Treatment of Asthma and/or Diabetes.

It is also believed that the molecules of this invention, alone or in combination with one or more peptides (as described in copending U.S. Ser. No. 10/649,378, filed Aug. 26, 2003) are effective in treating or prophylactically mitigating one or more symptoms of asthma and/or diabetes. Thus, in certain embodiments, this invention provides methods of mitigating a symptom of asthma and/or diabetes by administering to a mammal having the pathology or at risk for the pathology an amount of a small molecule of this invneiton sufficient to mitigate or prevent a symptom of the condition.

X. Small Molecule Administration.

The methods of this invention typically involve administering to an organism, preferably a mammal, more preferably a human one or more of the small molecules of this invention, optionally in combination with one or more of the peptides disclosed in U.S. Pat. No. 6,664,230, and/or PCT Applications WO 02/15923 and WO 2004/034977 and/or a lipid (e.g., as disclosed in WO 01/75168). The molecule(s) can be administered, as described herein, according to any of a number of standard methods including, but not limited to oral consumption, injection, suppository, nasal spray (e.g., oral inhalation or nasal inhalation), time-release implant, transdermal patch, and the like. In one particularly preferred embodiment, the small molecule(s) are administered orally (e.g. as a syrup, powder, drink, capsule, tablet, gelcap, etc.).

The methods can involve the administration of a single molecule of this invention or the administration of two or more different molecules. The molecules can be provided as monomers or in dimeric (e.g., linked), oligomeric or polymeric forms. In certain embodiments, the multimeric forms may comprise associated monomers (e.g. ionically or hydrophobically linked) while certain other multimeric forms comprise covalently linked monomers (directly linked or through a linker).

While the invention is described with respect to use in humans, it is also suitable for animal, e.g. veterinary use. Thus preferred organisms include, but are not limited to humans, non-human primates, canines, equines, felines, porcines, ungulates, largomorphs, and the like.

The methods of this invention are not limited to humans or non-human animals showing one or more symptom(s) of atherosclerosis (e.g., hypertension, plaque formation and rupture, reduction in clinical events such as heart attack, angina, or stroke, high levels of plasma cholesterol, high levels of low density lipoprotein, high levels of very low density lipoprotein, or inflammatory proteins such as CRP, etc.), but are useful in a prophylactic context. Thus, the small molecules of this invention (or mimetics thereof) may be administered to organisms to prevent the onset/development of one or more symptoms of atherosclerosis. Particularly preferred subjects in this context are subjects showing one or more risk factors for atherosclerosis (e.g. family history, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.).

The small molecules of this invention can also be administered to stimulate the formation and cycling of pre-beta high density lipoprotein-like particles and/or to promote reverse lipid transport and detoxification.

The small molecules are also useful for administration with statins where they enhance (e.g., synergize) the activity of the statin and permit the statin(s) to be administered at lower dosages and/or the anti-inflammatory properties of statins at any given dose are significantly enhanced.

In addition, the small molecules can be administered to reduce or eliminate one or more symptoms of osteoporosis and/or to prevent/inhibit the onset of one or more symptoms of osteoporosis.

XI. Pharmaceutical Formulations.

In order to carry out the methods of the invention, one or more small molecules of this invention (alone or in combination with therapeutic peptides (e.g., as disclosed in copending U.S. Ser. No. 10/649,378,) are administered, e.g., to an individual diagnosed as having one or more symptoms of atherosclerosis, or as being at risk for atherosclerosis or one or more of the other indications described herein (e.g., pathologies associated with an inflammatory response, osteoporosis, asthma, diabetes, etc.). The small molecule(s) can be administered in the “native” form or, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methods that typically involve reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of the peptides or mimetics are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups that can be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The small molecule(s) identified herein are useful for parenteral, topical, oral, nasal (or otherwise inhaled), rectal administration, local administration, and the like such as by aerosol or transdermally, for prophylactic and/or therapeutic treatment of atherosclerosis and/or symptoms thereof and/or for other indications as described herein. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The small molecule(s) of this invention are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

The excipients are preferably sterile and generally free of undesirable matter. These compositions may be sterilized by conventional, well-known sterilization techniques.

In therapeutic applications, the compositions of this invention are administered to a patient suffering from one or more symptoms of atherosclerosis or at risk for atherosclerosis and/or other indications described herein in an amount sufficient to cure or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of small molecule(s) can vary widely, and will be selected primarily based on fluid volumes, viscosities, body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. It will be appreciated that such dosages may be varied to optimize a therapeutic regimen in a particular subject or group of subjects.

In certain preferred embodiments, the small molecule(s) of this invention are administered orally (e.g. via a tablet) or as an injectable in accordance with standard methods well known to those of skill in the art. In other preferred embodiments, the small molecule(s), can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other preferred formulations for topical drug delivery include, but are not limited to, ointments and creams. Ointments are semisolid preparations, that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Certain preferred formulations are suitable for delivery by inhalation, e.g., through a nasal and/or oral inhaler. Typically such formulations are designed to be readily aerosolized and can be derivatized and/or complexed with excipients and/or protecting groups that increase uptake across an oral, nasal, bronchial mucosa and/or that increase stability during such uptake.

Unlike many therapeutics, the small molecule(s) of this invention can be administered, even orally, without protection against proteolysis by stomach acid, etc. Nevertheless, in certain embodiments, small molecule delivery can be enhanced by the use of protective excipients. This is typically accomplished either by complexing the small molecule(s) with a composition to render them resistant to acidic and enzymatic hydrolysis or by packaging the small molecule(s) in an appropriately resistant carrier such as a liposome. Means of protecting small molecule(s) for oral delivery are well known in the art (see, e.g., U.S. Pat. No. 5,391,377 describing lipid compositions for oral delivery of therapeutic agents).

A) Sustained Release Formulations.

Elevated serum half-life can be maintained by the use of sustained-release protein “packaging” systems. Such sustained release systems are well known to those of skill in the art. In one preferred embodiment, the ProLease biodegradable microsphere delivery system for proteins other molecules (Tracy (1998) Biotechnol. Prog. 14: 108; Johnson et al. (1996), Nature Med. 2: 795; Herbert et al. (1998), Pharmaceut. Res. 15, 357) a dry powder composed of biodegradable polymeric microspheres containing the active ingredient in a polymer matrix that can be compounded as a dry formulation with or without other agents.

The ProLease microsphere fabrication process was specifically designed to achieve a high encapsulation efficiency while maintaining integrity of the active ingredients. The process consists of (i) preparation of freeze-dried particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the drug, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the drug is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which the small molecule(s) are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

B) Combined Formulations.

In certain instances, one or more small molecule(s) of this invention are administered in conjunction with one or more active agents (e.g. statins, beta blockers, ACE inhibitors, lipids, etc.). The two agents (e.g., small molecule and statin) can be administered simultaneously or sequentially. When administered sequentially the two agents are administered so that both achieve a physiologically relevant concentration over a similar time period (e.g., so that both agents are active at some common time).

In certain embodiments, both agents are administered simultaneously. In such instances it can be convenient to provide both agents in a single combined formulation. This can be achieved by a variety of methods well known to those of skill in the art. For example, in a tablet formulation the tablet can comprise two layers one layer comprising, e.g. the statin(s), and the other layer comprising e.g. the small molecule(s). In a time release capsule, the capsule can comprise two time release bead sets, one for the small molecule(s) and one containing the statin(s).

The foregoing formulations and administration methods are intended to be illustrative and not limiting. It will be appreciated that, using the teaching provided herein, other suitable formulations and modes of administration can be readily devised.

XII. Additional Pharmacologically Active Agents.

Additional pharmacologically active agents may be delivered along with the primary active agents, e.g., the small molecule(s) of this invention. In one embodiment, such agents include, but are not limited to agents that reduce the risk of atherosclerotic events and/or complications thereof. Such agents include, but are not limited to beta blockers, beta blockers and thiazide diuretic combinations, statins, aspirin, ace inhibitors, ace receptor inhibitors (ARBs), and the like.

A) Statins.

It is believed that administration of one or more small molecule(s) of this invention “concurrently” with one or more statins synergistically enhances the effect of the statin(s). That is, the statins can achieve a similar efficacy at lower dosage thereby obviating potential adverse side effects (e.g. muscle wasting) associated with these drugs and/or cause the statins to be significantly more anti-inflammatory at any given dose.

The major effect of the statins is to lower LDL-cholesterol levels, and they lower LDL-cholesterol more than many other types of drugs. Statins generally inhibit an enzyme, HMG-CoA reductase, which controls the rate of cholesterol production in the body. These drugs typically lower cholesterol by slowing down the production of cholesterol and by increasing the liver's ability to remove the LDL-cholesterol already in the blood.

The large reductions in total and LDL-cholesterol produced by these drugs appears to result in large reductions in heart attacks and heart disease deaths. Thanks to their track record in these studies and their ability to lower LDL-cholesterol, statins have become the drugs most often prescribed when a person needs a cholesterol-lowering medicine. Studies using statins have reported 20 to 60 percent lower LDL-cholesterol levels in patients on these drugs. Statins also reduce elevated triglyceride levels and produce a modest increase in HDL-cholesterol. Recently it has been appreciated that statins have anti-inflammatory properties that may not be directly related to the degree of lipid lowering achieved. For example it has been found that statins decrease the plasma levels of the inflammatory marker CRP relatively independent of changes in plasma lipid levels. This anti-inflammatory activity of statins has been found to be as or more important in predicting the reduction in clinical events induced by statins than is the degree of LDL lowering.

The statins are usually given in a single dose at the evening meal or at bedtime. These medications are often given in the evening to take advantage of the fact that the body makes more cholesterol at night than during the day. When combined with the small molecule(s) described herein, the combined small molecule/statin treatment regimen will also typically be given in the evening.

Suitable statins are well known to those of skill in the art. Such statins include, but are not limited to atorvastatin (Lipitor®, Pfizer), simvastatin (Zocor®, Merck0, pravastatin (Pravachol®, Bristol-Myers Squibb®, fluvastatin (Lescol®, Novartis), lovastatin (Mevacor®, Merck), rosuvastatin (Crestor®, Astra Zeneca), and Pitavastatin (Sankyo), and the like.

The combined statin/small molecule dosage can be routinely optimized for each patient. Typically statins show results after several weeks, with a maximum effect in 4 to 6 weeks. Prior to combined treatment with a statin and one of the small molecules described herein, the physician would obtain routine tests for starting a statin including LDL-cholesterol and HDL-cholesterol levels. Additionally, the physician would also measure the anti-inflammatory properties of the patient's HDL and determine CRP levels with a high sensitivity assay. After about 4 to 6 weeks of combined treatment, the physician would typically repeat these tests and adjust the dosage of the medications to achieve maximum lipid lowering and maximum anti-inflammatory activity.

B) Cholesterol Absorption Inhibitors.

In certain embodiments, one or more small molecules of this invention are administered to a subject in conjunction with one or more cholesterol absorption inhibitors. The small molecule(s) can be administered before, after, or simultaneously with the cholesterol absorption inhibitor. In the latter case, the cholesterol absorption inhibitor can be provided as a separate formulation or as a combined formulation with one or more of the small molecule(s).

Cholesterol absorption inhibitors are well known to those of skill in the art. One important cholesterol absorption inhibitor is Ezetimibe, also known as 1-(4-fluorophenyl)-3(R)-[3-(4-fluorophenyl)-3(S)-hydroxypropyl]-4(S)-(4-hydroxyphenyl)-2-azetidinone (available from Merck). Ezetimibe reduces blood cholesterol by inhibiting the absorption of cholesterol by the small intestine.

C) Beta Blockers.

Suitable beta blockers include, but are not limited to cardioselective (selective beta 1 blockers), e.g., acebutolol (Sectral™), atenolol (Tenormin™), betaxolol (Kerlone™), bisoprolol (Zebeta™), metoprolol (Lopressor™), and the like. Suitable non-selective blockers (block beta 1 and beta 2 equally) include, but are not limited to carteolol (Cartrol™), nadolol (Corgard™), penbutolol (Levatol™), pindolol (Visken™), carvedilol, (Coreg™), propranolol (Inderal™), timolol (Blockadren™), labetalol (Normodyne™, Trandate™), and the like.

Suitable beta blocker thiazide diuretic combinations include, but are not limited to Lopressor HCT, ZIAC, Tenoretic, Corzide, Timolide, Inderal LA 40/25, Inderide, Normozide, and the like.

D) ACE Inhibitors.

Suitable ace inhibitors include, but are not limited to captopril (e.g. Capoten™ by Squibb), benazepril (e.g., Lotensin™ by Novartis), enalapril (e.g., Vasotec™ by Merck), fosinopril (e.g., Monopril™ by Bristol-Myers), lisinopril (e.g. Prinivil™ by Merck or Zestril™ by Astra-Zeneca), quinapril (e.g. Accupril™ by Parke-Davis), ramipril (e.g., Altace™ by Hoechst Marion Roussel, King Pharmaceuticals), imidapril, perindopril erbumine (e.g., Aceon™ by Rhone-Polenc Rorer), trandolapril (e.g., Mavik™ by Knoll Pharmaceutical), and the like. Suitable ARBS (Ace Receptor Blockers) include but are not limited to losartan (e.g. Cozaar™ by Merck), irbesartan (e.g., Avapro™ by Sanofi), candesartan (e.g., Atacand™ by Astra Merck), valsartan (e.g., Diovan™ by Novartis), and the like.

E) Lipid-Based Formulations.

In certain embodiments, the small molecule(s) of this invention are administered in conjunction with one or more lipids. The lipids can be formulated as an active agent, and/or as an excipient to protect and/or enhance transport/uptake of the small molecule(s) or they can be administered separately.

Without being bound by a particular theory, it was discovered of this invention that administration (e.g. oral administration) of certain phospholipids can significantly increase HDL/LDL ratios. In addition, it is believed that certain medium-length phospholipids are transported by a process different than that involved in general lipid transport. Thus, co-administration of certain medium-length phospholipids with the small molecule(s) of this invention confer a number of advantages: They protect the phospholipids from digestion or hydrolysis, they improve small molecule uptake, and they improve HDL/LDL ratios.

The lipids can be formed into liposomes that encapsulate the polypeptides of this invention and/or they can be simply complexed/admixed with the polypeptides. Methods of making liposomes and encapsulating reagents are well known to those of skill in the art (see, e.g., Martin and Papahadjopoulos (1982) J. Biol. Chem., 257: 286-288; Papahadjopoulos et al. (1991) Proc. Natl. Acad. Sci. USA, 88: 11460-11464; Huang et al. (1992) Cancer Res., 52:6774-6781; Lasic et al. (1992) FEBS Lett., 312: 255-258., and the like).

Preferred phospholipids for use in these methods have fatty acids ranging from about 4 carbons to about 24 carbons in the sn-1 and sn-2 positions. In certain preferred embodiments, the fatty acids are saturated. In other preferred embodiments, the fatty acids can be unsaturated. Various preferred fatty acids are illustrated in Table 2.

TABLE 2
Preferred fatty acids in the sn-1 and/or sn-2 position of the
preferred phospholipids for administration of D polypeptides.

| Carbon No. | Common Name | IUPAC Name |
|---|---|---|
| 3:0 | Propionoyl | Trianoic |
| 4:0 | Butanoyl | Tetranoic |
| 5:0 | Pentanoyl | Pentanoic |
| 6:0 | Caproyl | Hexanoic |
| 7:0 | Heptanoyl | Heptanoic |
| 8:0 | Capryloyl | Octanoic |
| 9:0 | Nonanoyl | Nonanoic |
| 10:0 | Capryl | Decanoic |
| 11:0 | Undcanoyl | Undecanoic |
| 12:0 | Lauroyl | Dodecanoic |
| 13:0 | Tridecanoyl | Tridecanoic |
| 14:0 | Myristoyl | Tetradecanoic |
| 15:0 | Pentadecanoyl | Pentadecanoic |
| 16:0 | Palmitoyl | Hexadecanoic |
| 17:0 | Heptadecanoyl | Heptadecanoic |
| 18:0 | Stearoyl | Octadecanoic |
| 19:0 | Nonadecanoyl | Nonadecanoic |
| 20:0 | Arachidoyl | Eicosanoic |
| 21:0 | Heniecosanoyl | Heniecosanoic |
| 22:0 | Behenoyl | Docosanoic |
| 23:0 | Trucisanoyl | Trocosanoic |
| 24:0 | Lignoceroyl | Tetracosanoic |
| 14:1 | Myristoleoyl (9-cis) | |
| 14:1 | Myristelaidoyl (9-trans) | |
| 16:1 | Palmitoleoyl (9-cis) | |
| 16:1 | Palmitelaidoyl (9-trans) | |

The fatty acids in these positions can be the same or different. Particularly preferred phospholipids have phosphorylcholine at the sn-3 position.
XIII. Kits.

In another embodiment this invention provides kits for amelioration of one or more symptoms of atherosclerosis and/or for the prophylactic treatment of a subject (human or animal) at risk for atherosclerosis and/or for stimulating the formation and cycling of pre-beta high density lipoprotein-like particles and/or for inhibiting one or more symptoms of osteoporosis. The kits preferably comprise a container containing one or more of the small molecules of this invention. The small molecule can be provided in a unit dosage formulation (e.g., suppository, tablet, caplet, patch, etc.) and/or may be optionally combined with one or more pharmaceutically acceptable excipients.

The kit can, optionally, further comprise one or more other agents used in the treatment of heart disease and/or atherosclerosis and/or one or more of the other indications described herein. Such agents include, but are not limited to, beta blockers, vasodilators, aspirin, statins, ace inhibitors or ace receptor inhibitors (ARBs) and the like, e.g., as described above.

In certain preferred embodiments, the kits additionally include a statin (e.g. cerivastatin, atorvastatin, simvastatin, pravastatin, fluvastatin, lovastatin. rosuvastatin, pitavastatin, etc.) either formulated separately or in a combined formulation with the peptide(s). Typically the dosage of a statin in such a formulation can be lower than the dosage of a statin typically presecribed without the synergistic peptide.

In addition, the kits optionally include labeling and/or instructional materials providing directions (i.e., protocols) for the practice of the methods or use of the “therapeutics” or “prophylactics” of this invention. Preferred instructional materials describe the use of one or more small molecule of this invention to mitigate one or more symptoms of atherosclerosis (or other indications described herein) and/or to prevent the onset or increase of one or more of such symptoms in an individual at risk for atherosclerosis and/or to stimulate the formation and cycling of pre-beta high density lipoprotein-like particles and/or to inhibit one or more symptoms of osteoporosis. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Physical Properties of Novel Small Organic Molecules that Predict Ability to Render HDL More Anti-Inflammatory and Mitigate Atherosclerosis in a Mammal

It was a surprising finding of this invention that a number of physical properties predict the ability of the small molecules of this invention to render HDL more anti-inflammatory and to mitigate atherosclerosis and/or other pathologies characterized by an inflammatory response in a mammal. The physical properties include high solubility in ethyl acetate (e.g., greater than about 4 mg/mL), and solubility in aqueous buffer at pH 7.0. In addition, upon contacting phospholipids such as 1,2-Dimyristoyl-sn-glycero-3-phosphocholine (DMPC), in an aqueous environment, the particularly effective small molecules form or participate in the formation of particles with a diameter of approximately 7.5 nm (±0.1 nm), and/or form or participate in the formation of stacked bilayers with a bilayer dimension on the order of 3.4 to 4.1 nm with spacing between the bilayers in the stack of approximately 2 nm, and/or also form or participate in the formation of vesicular structures of approximately 38 nm). In certain preferred embodiments, the small peptides have a molecular weight of less than about 900 Da.

The predictive effect of these physical properties is illustrated by a comparison of two peptides of which certain small molecules described herein are analogues. The first peptide is Boc-Lys(εBoc)-Glu-Arg-Ser(tBu)-OtBu (SEQ ID NO:3), corresponds to SEQ ID NO:254 in copending application U.S. Ser. No. 10/649,378), while the second peptide is Boc-Lys(εBoc)-Arg-Glu-Ser(tBu)-OtBu (SEQ ID NO:4), corresponds to SEQ ID NO:258 in U.S. Ser. No. 10/649,378. While this example describes the results obtained with small peptides, it is believed the same results will obtain with the small molecules of the present invention.

To evaluate solubility in ethyl acetate, each peptide was weighed and added to a centrifuge tube and ethyl acetate (HPLC grade; residue after evaporation <0.0001%) was added to give a concentration of 10 mg/mL. The tubes were sealed, vortexed and kept at room temperature for 30 minutes with vortexing every 10 minutes. The tubes were then centrifuged for 5 minutes at 10,000 rpm and the supernatant was removed to a previously weighed tube. The ethyl acetate was evaporated under argon and the tubes weighed to determine the amount of peptide that had been contained in the supernatant. The percent of the originally added peptide that was dissolved in the supernatant is shown on the Y-axis. he data are mean±S.D. Control represents sham treated tubes; SEQ ID NO:3 and SEQ ID NO:4 were both synthesized from all D-amino acids. The sequence Boc-Phe-Arg-Glu-Leu-OtBu (SEQ ID NO:5, SEQ ID NO:250 in copending application U.S. Ser. No. 10/649,378) was synthesized from all L-amino acids.

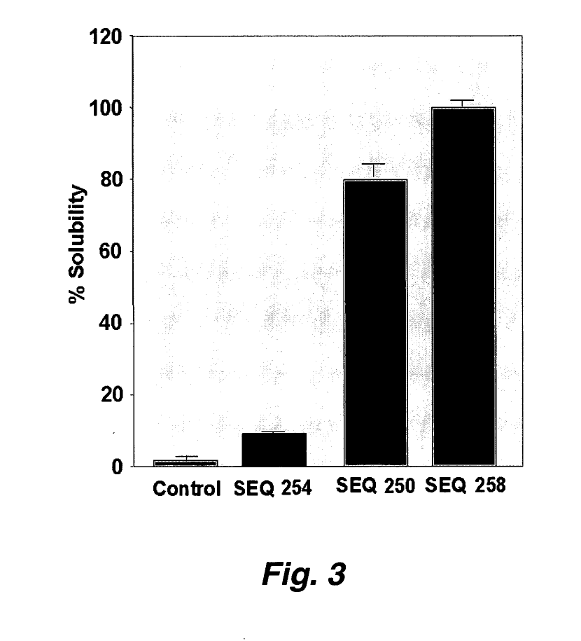

As shown in FIG. 3, SEQ ID NO: 4 is very soluble in ethyl acetate while SEQ ID NO:3 is not (both synthesized from all D-amino acids). Additionally the data in FIG. 3 demonstrate that SEQ ID NO:5 [Boc-Phe-Arg-Glu-Leu-OtBu] (synthesized from all L-amino acids) is also very soluble in ethyl acetate.

To 1 mg/ml of DMPC suspension in phosphate buffered saline (PBS) was added 10% deoxycholate until the DMPC was dissolved. Peptides, SEQ ID NO:4 or SEQ ID NO:3, were added (DMPC: peptide; 1:10; wt:wt) and the reaction mixture dialyzed. After dialysis the solution remained clear with SEQ ID NO:4 but was turbid after the deoxycholate was removed by dialysis in the case of SEQ ID NO:3.

FIGS. 4-6—demonstrate that when SEQ ID NO:4 was added to DMPC in an aqueous environment particles with a diameter of approximately 7.5 nm formed, stacked lipid bilayers with a bilayer dimension on the order of 3.4 to 4.1 nm with spacing between the bilayers in the stack of approximately 2 nm formed, and vesicular structures of approximately 38 nm also formed.

In particular, FIG. 4 shows an electron micrograph prepared with negative staining and at 147,420× magnification. The arrows indicate SEQ ID NO:4 particles measuring 7.5 nm (they appear as small white particles).

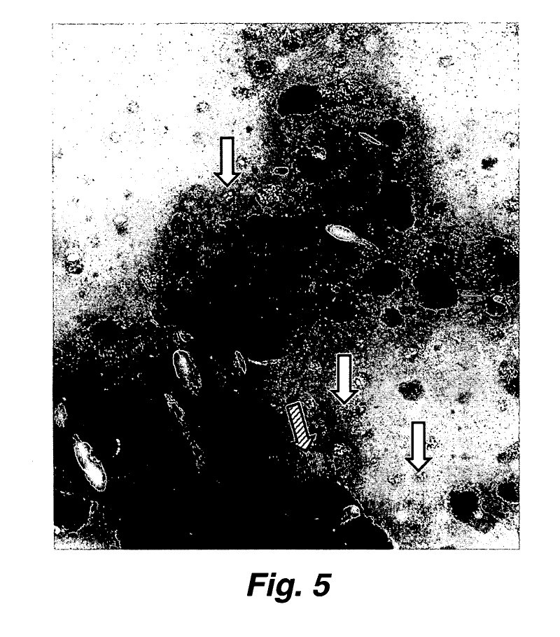

As illustrated in FIG. 5 a peptide comprising SEQ ID NO:4 added to DMPC in an aqueous environment forms particles with a diameter of approximately 7.5 nm (white arrows), and stacked lipid-peptide bilayers (striped arrows pointing to the white lines in the cylindrical stack of disks) with a bilayer dimension on the order of 3.4 to 4.1 nm with spacing between the bilayers (black lines between white lines in the stack of disks) of approximately 2 nm.

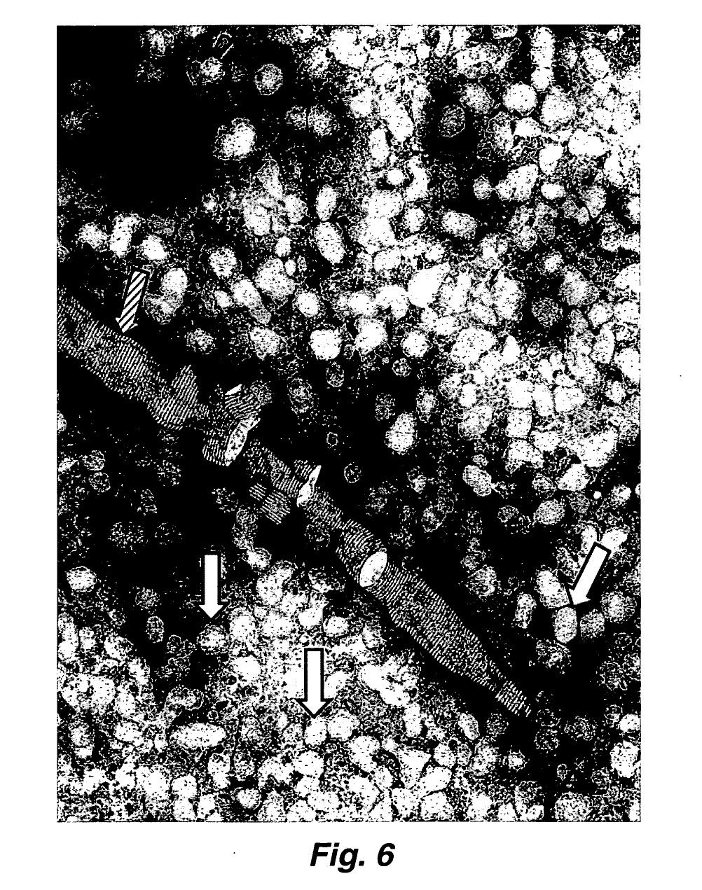

FIG. 6 shows that the peptide of SEQ ID NO:4 added to DMPC in an aqueous environment forms stacked lipid-peptide bilayers (striped arrow) and vesicular structures of approximately 38 nm white arrows).

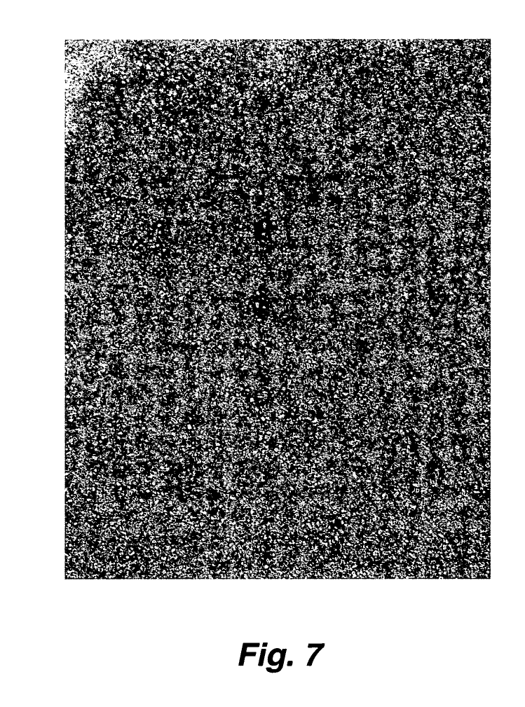

FIG. 7 shows that DMPC in an aqueous environment without SEQ ID NO: 4 does not form particles with a diameter of approximately 7.5 nm, or stacked lipid-petide bilayers, nor vesicular structures of approximately 38 nm.

The peptide of SEQ ID NO:3 (which differs from the peptide of SEQ ID NO: 4 only in the order of arginine and glutamic acid in regard to the amino and carboxy termini of the peptide) did not form particles with a diameter of approximately 7.5 nm, or stacked lipid-peptide bilayers, nor vesicular structures of approximately 38 nm under the conditions as described in FIG. 4 (data not shown). Thus, the order of arginine and glutamic acid in the peptide dramatically altered its ability to interact with DMPC and this was predicted by the solubility in ethyl acetate (i.e., the peptide of SEQ ID NO: 4 was highly soluble in ethyl acetate and formed particles with a diameter of approximately 7.5 nm, and stacked lipid-peptide bilayers, as well as vesicular structures of approximately 38 nm, while the peptide of SEQ ID NO:3 was poorly soluble in ethyl acetate and did not form these structures under the conditions described in FIG. 4). In addition to the protocol described in FIG. 4, similar results were also obtained if the DMPC suspension in PBS was added to the peptide of SEQ ID NO: 4 (DMPC:peptide; 1:10; wt:wt) or to the peptide of SEQ ID NO:3 (DMPC:peptide; 1:10; wt:wt) and the mixture recycled between just above the transition temperature of DMPC (just above 50° C.) and room temperature each hour for several cycles and then left at room temperature for 48 hours (data not shown).

The physical properties of the peptide of SEQ ID NO: 4 (but not the peptide of SEQ ID NO:3) indicate that this peptide has amphipathic properties (i.e., it is highly soluble in ethyl acetate, it is also soluble in aqueous buffer at pH 7.0 [data not shown], and it interacts with DMPC as described above). It was a surprising finding of this invention that the peptides that are highly soluble in ethyl acetate, and are also soluble in aqueous buffer at pH 7.0, interacted with DMPC to form lipid-peptide complexes that are remarkably similar to the nascent HDL particles formed by the interaction of apoA-I with cells (Forte, et al. (1993) J. Lipid Res. 34: 317-324).

Table 3 compares the interaction of lipid-free human apoA-I with CHO—C19 cells in vitro with the interaction of SEQ ID NO: 4 with DMPC as indicated in FIGS. 4-7 above.

TABLE 3
Comparison of the interaction of the peptide of SEQ ID NO: 4
with DMPC as indicated in FIG. 4-7 above with the interaction
of lipid-free human apoA-I interacting with CHO—C-19 cells as
described in Forteet al. (1993) J. Lipid Res. 34: 317-324.

| | Property | |
|---|---|---|
| | | SEQ ID NO: |
| | ApoA-I/Cells | 6/DMPC |
| | Prominent Feature | |
| | Discoidal particles | |
| | stacked in rouleaux | Stacked bilayers in |
| | formation | cylindrical form |
| Bilayer dimension | 4.6 nm | 3.4-4.1 nm |
| Spacing between discoidal | 1.9 nm | 2.0 nm |
| particles/bilayers | | |
| Size “Nascent HDL Particles” | 7.3 nm | 7.5 nm |
| Vesicular structures | 34.7 nm | 38 nm |

Thus, the small peptides described here that are highly soluble in ethyl acetate and are also soluble in aqueous buffers at pH 7.0 interact with lipids (DMPC) similar to apoA-I, which has a molecular weight of 28,000 Daltons.

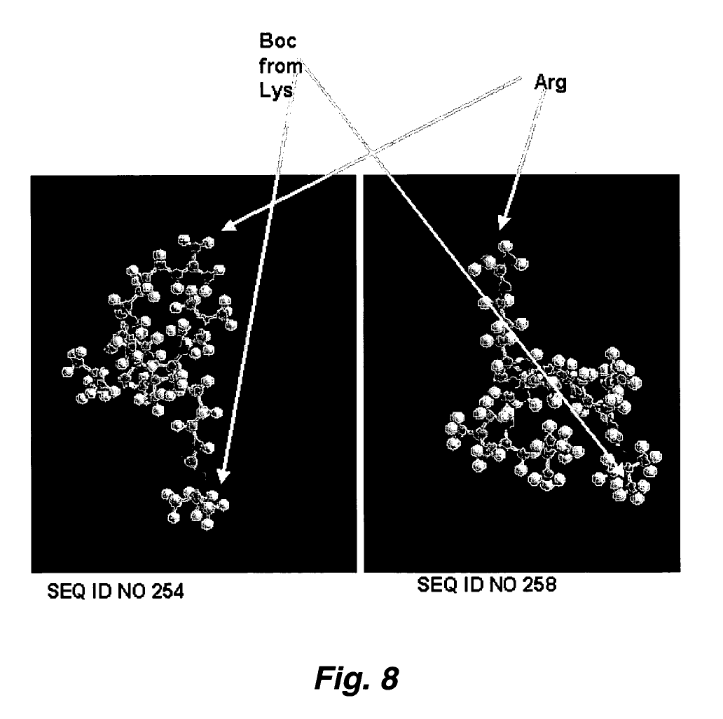

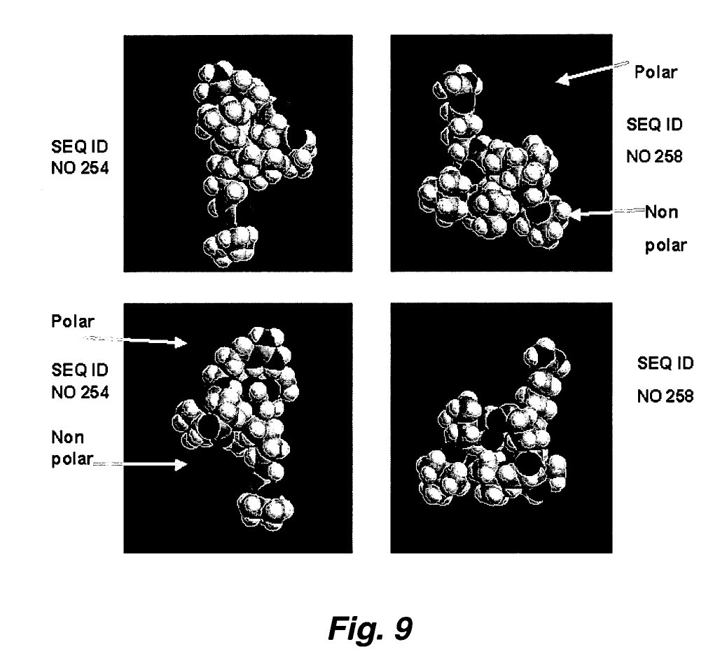

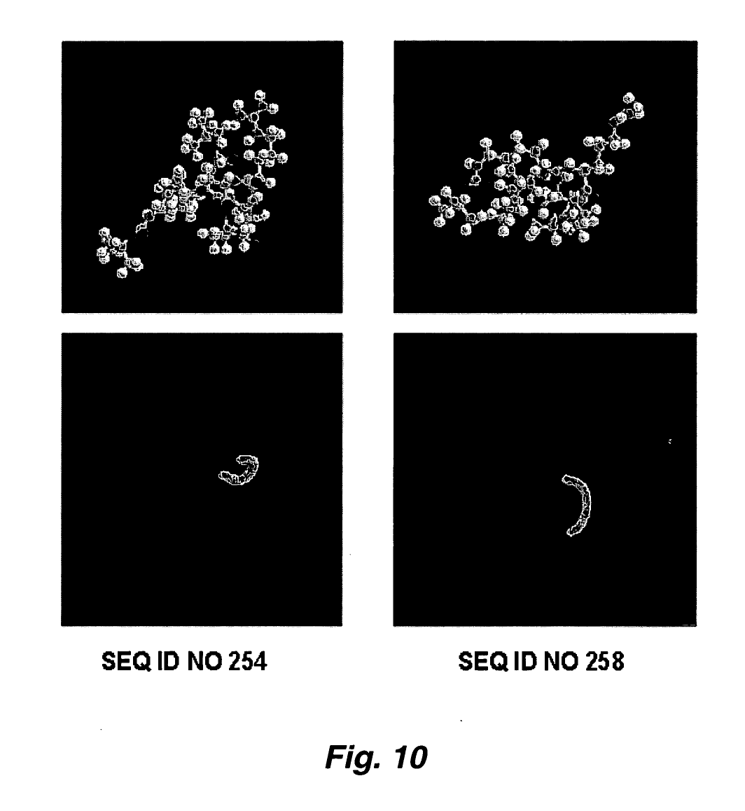

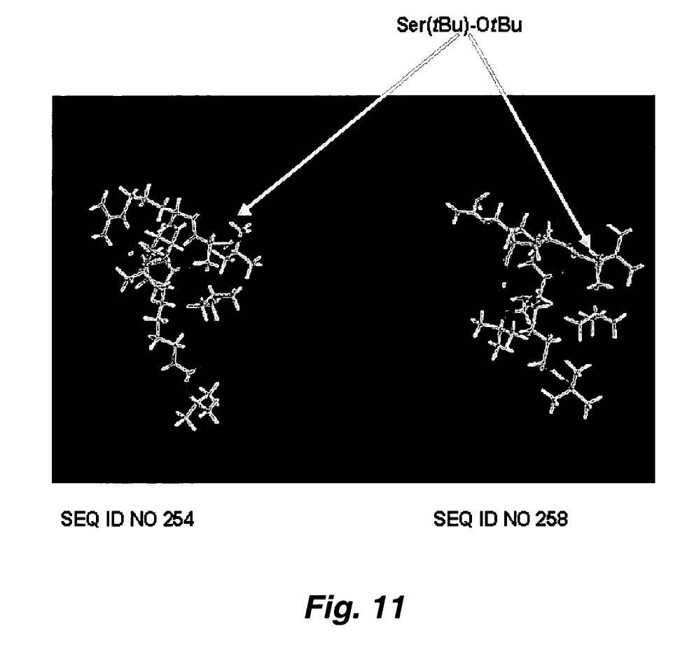

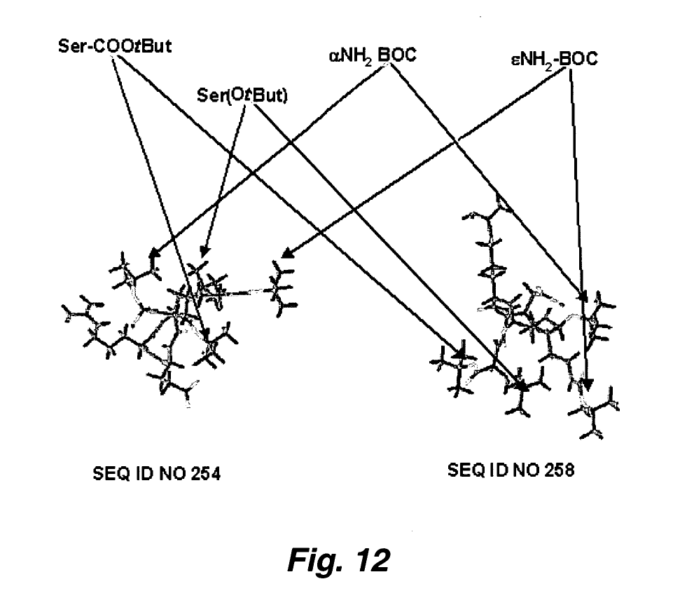

The molecular models shown in FIGS. 8-12 demonstrate the spatial characteristics of SEQ ID NO:3 compared to SEQ ID NO: 4.

The molecular models shown in FIGS. 8-12 indicate that both the peptide of SEQ ID NO:3 and the peptide of SEQ ID NO: 4 contain polar and non-polar portions in each molecule but there are spatial differences in the arrangement of the polar and non-polar components of the two molecules. As a result of the differences in the spatial arrangement of the molecules there are differences in the solubility of the two molecules in ethyl acetate (FIG. 3) and in their interaction with DMPC (FIGS. 4-7).

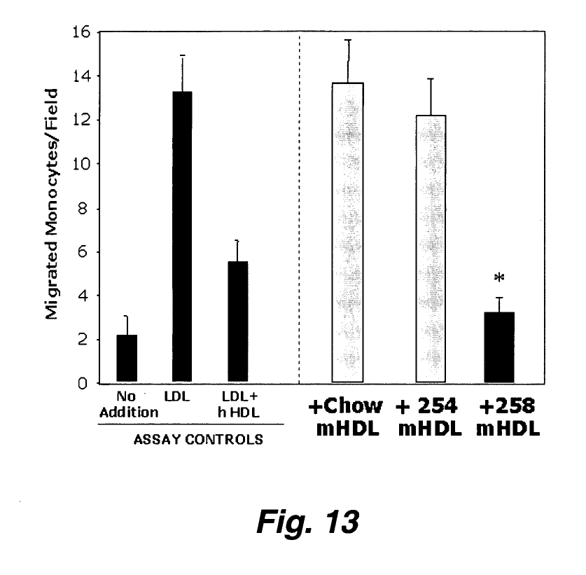

The data in FIGS. 13-15 demonstrate that the physical properties of the peptide of SEQ ID NO:3 versus the peptide of SEQ ID NO: 4 predict the ability of these molecules to render HDL anti-inflammatory and mitigate atherosclerosis when given orally to a mammal.

Female apoE null mice at age 8 weeks were given no additions to their diet (Chow) or received 200 μg/gm chow of SEQ ID NO:3 (+254) or 200 μg/gm chow of SEQ ID NO: 4 (+258), both synthesized from all D-amino acids. After 15 weeks the mice were bled and their plasma fractionated by FPLC and their HDL (mHDL) tested in a human artery wall cell coculture. A standard human LDL (at 100 μg/mL of LDL-cholesterol) was added alone (LDL) or not added (no addition) or was added with 50 μg/mL of normal human HDL (hHDL) or 50 μg/mL of mouse HDL (mHDL) to human artery wall cocultures and the resulting monocyte chemotactic activity was determined and plotted on the Y-axis. FIG. 13 shows that the HDL from apoE null mice was rendered anti-inflammatory after the mice were fed SEQ ID NO: 4 but not after SEQ ID NO:3.

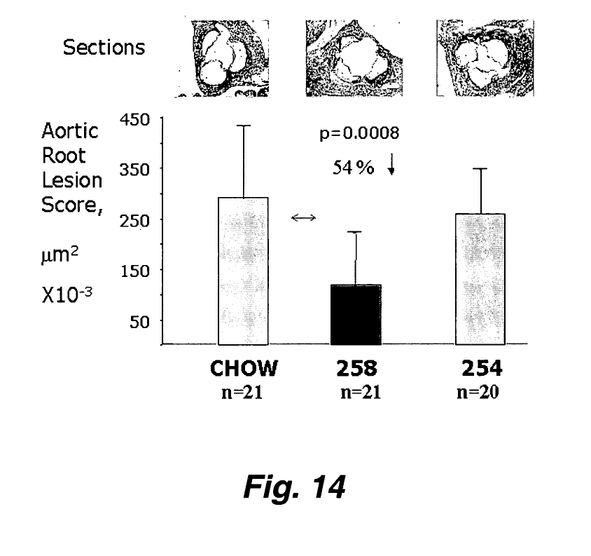

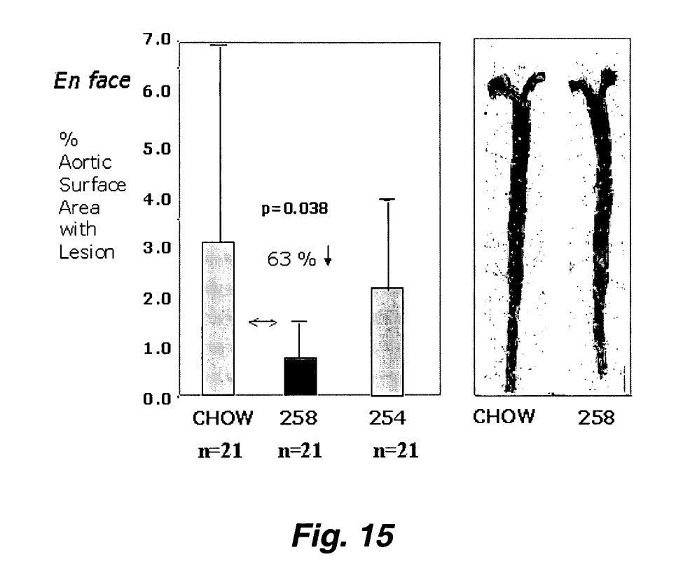

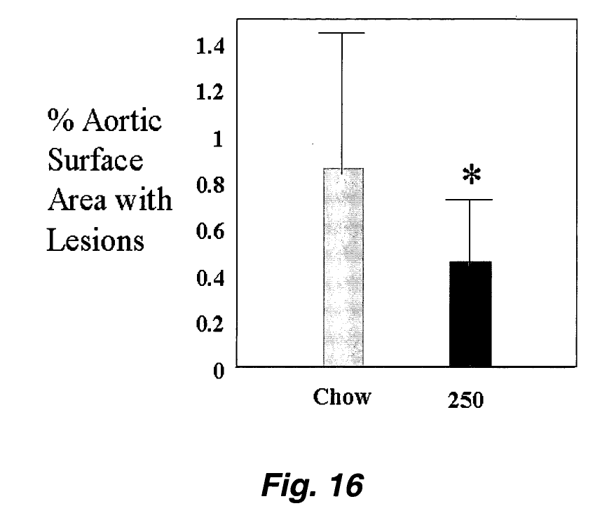

As shown in FIG. 14 the peptide of SEQ ID NO: 4 but not the peptide of SEQ ID NO:3 significantly reduced atherosclerosis in the aortic root (aortic sinus) of the apoE null mice described above. FIG. 15 demonstrates that SEQ ID NO: 4 but not SEQ ID NO:3 also significantly decreased atherosclerosis in en face preparations of the aortas. FIG. 3 demonstrates that the solubility in ethyl acetate of the peptide of SEQ ID NO:5 synthesized from all L-amino acids (see FIG. 3 above) accurately predicts the ability of this molecule to ameliorate atherosclerosis in apoE null mice.

Thus, the physical properties of these small peptides accurately predicted the ability of the peptides to ameliorate atherosclerosis in apoE null mice.

We thus teach that small peptides, typically with molecular weights of less than about 900 Daltons that are highly soluble in ethyl acetate (greater than about 4 mg/mL), and also are soluble in aqueous buffer at pH 7.0, and that when contacted with phospholipids such as 1,2-Dimyristoyl-sn-glycero-3-phosphocholine (DMPC), in an aqueous environment, form particles with a diameter of approximately 7.5 nm, and/or form stacked bilayers with a bilayer dimension on the order of 3.4 to 4.1 nm with spacing between the bilayers in the stack of approximately 2 nm, and/or they also form vesicular structures of approximately 38 nm, when administered to a mammal render HDL more anti-inflammatory and mitigate one or more symptoms of atherosclerosis and other pathologies characterized by an inflammatory response.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A small molecule that ameliorates one or more symptoms of a pathology characterized by an inflammatory response in a mammal, wherein said small molecule:
is soluble in ethyl acetate at a concentration greater than 4 mg/mL;
is soluble in aqueous buffer at pH 7.0;
when contacted with a phospholipid in an aqueous environment, forms particles with a diameter of approximately 7.5 nm and forms stacked bilayers with a bilayer dimension on the order of 3.4 to 4.1 nm with spacing between the bilayers in the stack of approximately 2 nm; and
has a molecular weight les than 900 daltons.

2. The small molecule of claim 1, wherein said molecule induces the conversion of pro-inflammatory HDL to anti-inflammatory HDL or makes anti-inflammatory HDL more anti-inflammatory.

3. The small molecule of claim 1, wherein said molecule protects a phospholipid against oxidation by an oxidizing agent.

4. The small molecule of claim 3, wherein said oxidizing agent is selected from the group consisting of hydrogen peroxide, 13(S)-HPODE, 15(S)-HPETE, HPODE, HPETE, HODE, and HETE.

5. The small molecule of claim 3, wherein said phospholipid is selected from the group consisting of 1-palmitoyl-2-arachidonoyl-sn-glycero-3-phosphorylcholine (PAPC), 1-stearoyl-2-arachidonoyl-sn-glycero-3-phosphorylcholine (SAPC)), and 1-stearoyl-2-arachidonyl-sn-glycero-3-phosphorylethanolamine (SAPE).

6. The small molecule of claim 1, wherein said small molecule has the formula: 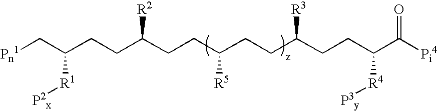
wherein:
P¹, P², P³, and P⁴ are independently selected hydrophobic protecting groups;
R¹ and R⁴ are independently selected amino acid R groups;
n, i, x, y, and z are independently zero or 1 and
when n and x are both zero, R¹ is a hydrophobic group;
when y and i are both zero, R⁴ is a hydrophobic group;
R² and R³ are acidic or basic groups at pH 7.0 such that when R² is acidic, R³ is basic and when R is basic, R is acidic; and
R⁵, when present is selected from the group consisting of an aromatic group, an aliphatic group, a postively charged group, and a negatively charged group.

7. The small molecule of claim 6, wherein R² or R³ is —(CH₂)j-COOH where j=1, 2, 3, or 4.

8. The small molecule of claim 6, wherein R² or R³ is —(CH₂)j-NH₂ where j=1, 2, 3, 4, or 5, or —(CH₂)j-NH—C(═NH)—NH₂ where n=1, 2, 3 or 4.

9. The small molecule of claim 6, wherein R², R³, and R⁵, when present, are amino acid R groups.

10. The small molecule of claim 6, wherein R² and R³ are independently selected from the group consisting of an aspartic acid R group, a glutamic acid R group, a lysine R group, a histidine R group, and an arginine R group.

11. The small molecule of claim 6, wherein R1 is selected from the group consisting of a Lys R group, a Trp R group, a Phe R group, a Leu R group, an Orn R group, and a norLeu R group.

12. The small molecule of claim 6, wherein R4 is selected from the group consisting of a Ser R group, a Thr R group, an Ile R group, a Leu R group, a norLeu R group, a Phe R group, and a Tyr R group.

13. The small molecule of claim 6, wherein x is 1, and R5 is an aromatic group.

14. The small molecule of claim 6, wherein x is 1, and R⁵ is a Trp R group.

15. The small molecule of claim 6, wherein at least one of n, x, y, and i is 1 and P¹, P², P³, and P⁴ when present, are independently selected from the group consisting of polyethylene glycol (PEG), an acetyl, amide, 3 to 20 carbon alkyl groups, Fmoc, 9-fluoreneacetyl group, 1-fluorenecarboxylic group, 9-fluorenecarboxylic, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), Benzyloxymethyl (Bom), t-butoxycarbonyl (Boc), cyclohexyloxy (cHxO), t-butoxymethyl (Bum), t-butoxy (tBuO), t-Butyl (tBu), a propyl group, a butyl group, a pentyl group, a hexyl group, and trifluoroacetyl (TFA).

16. The small molecule of claim 15, wherein P1 when present and/or P2 when present are independently selected from the group consisting of Boc-, Fmoc-, and Nicotinyl-.

17. The small molecule of claim 15, wherein P3 when present and/or P4 when present are independently selected from the group consisting of tBu, and OtBu.

18. The small molecule of claim 6, wherein z is zero and said molecule has the formula:

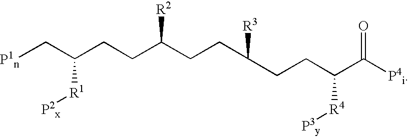

19. The small molecule of claim 14, wherein R2 and R3 are amino acid R groups.

20. The small molecule of claim 14, wherein R2 and R3 are independently selected from the group consisting of an aspartic acid R group, a glutamic acid R group, a lysine R group, a histidine R group, and an arginine R group.

21. The small molecule of claim 14, wherein R1 is selected from the group consisting of a Lys R group, a Trp R group, a Phe R group, a Leu R group, an Orn R group, and a norLeu R group.

22. The small molecule of claim 14, wherein R4 is selected from the group consisting of a Ser R group, a Thr R group, an Ile R group, a Leu R group, a norLeu R group, a Phev, and a Tyr R group.

23. The small molecule of claim 14, wherein at least one of n, x, y, and i is 1 and P1, P2, P3, and P4 when present, are independently selected from the group consisting of polyethylene glycol (PEG), an acetyl, amide, 3 to 20 carbon alkyl groups, Fmoc, 9-fluoreneacetyl group, 1-fluorenecarboxylic group, 9-fluorenecarboxylic, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), Benzyloxymethyl (Bom), t-butoxycarbonyl (Boc), cyclohexyloxy (cHxO), t-butoxymethyl (Bum), t-butoxy (tBuO), t-Butyl (tBu), a propyl group, a butyl group, a pentyl group, a hexyl group, and trifluoroacetyl (TFA).

24. The small molecule of claim 23, wherein P1 when present and/or P2 when present are independently selected from the group consisting of Boc-, Fmoc-, and Nicotinyl-.

25. The small molecule of claim 23, wherein P3 when present and/or P4 when present are independently selected from the group consisting of tBu, and OtBu.

26. The small molecule of claim 6, wherein z is zero and said molecule has the formula:

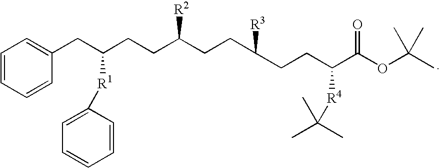

27. The small molecule of claim 26, wherein R2 and R3 are amino acid R groups.

28. The small molecule of claim 26, wherein R2 and R3 are independently selected from the group consisting of an aspartic acid R group, a glutamic acid R group, a lysine R group, a histidine R group, and an arginine R group.

29. The small molecule of claim 26, wherein R1 is selected from the group consisting of a Lys R group, a Trp R group, a Phe R group, a Leu R group, an Orn R group, and a norLeu R group.

30. The small molecule of claim 26, wherein R4 is selected from the group consisting of a Ser R group, a Thr R group, an Ile R group, a Leu R group, a norLeu R group, a Phev, and a Tyr R group.

31. The small molecule of claim 26, wherein said molecule has the formula:

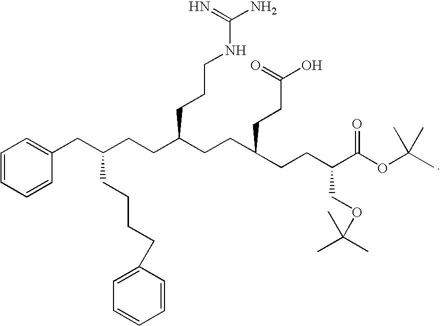

32. The small molecule of claim 6, wherein said pathology is selected from the group consisting of atherosclerosis, rheumatoid arthritis, lupus erythematous, polyarteritis nodosa, osteoporosis, Altzheimer's disease and a viral illnesses.

33. A small molecule that ameliorates one or more symptoms of a pathology characterized by an inflammatory response in a mammal, said small molecule having the formula: 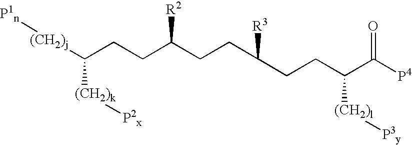
wherein:
P¹, P², P³, and P⁴ are independently selected hydrophobic protecting groups;
n, x, and y are independently zero or 1;
j, k, and l are independently zero, 1, 2, 3, 4, or 5; and
R² and R³ are acidic or basic groups at pH 7.0 such that when R² is acidic, R³ is basic and when R² is basic, R³ is acidic;
said small molecule is soluble in water; and
said small molecule has a molecular weight less than about 900 Daltons.

34. The small molecule of claim 33, wherein said molecule induces the conversion of pro-inflammatory HDL to anti-inflammatory HDL or makes anti-inflammatory HDL more anti-inflammatory.

35. The small molecule of claim 33, wherein protects a phospholipid against oxidation by an oxidizing agent

36. The small molecule of claim 35, wherein said oxidizing agent is selected from the group consisting of hydrogen peroxide, 13(S)-HPODE, 15(S)-HPETE, HPODE, HPETE, HODE, and HETE.

37. The small molecule of claim 35, wherein said phospholipid is selected from the group consisting of 1-palmitoyl-2-arachidonoyl-sn-glycero-3-phosphorylcholine (PAPC), 1-stearoyl-2-arachidonoyl-sn-glycero-3-phosphorylcholine (SAPC)), and 1-stearoyl-2-arachidonyl-sn-glycero-3-phosphorylethanolamine (SAPE).

38. The small molecule of claim 33, wherein:
n, x, y, j, and l are 1; and
k is 4.

39. The small molecule of claim 33, wherein P1 and P2 are aromatic protecting groups.

40. The small molecule of claim 33, wherein R2 and R3 are amino acid R groups.

41. The small molecule of claim 33, wherein R2 and R3 are independently selected from the group consisting of an aspartic acid R group, a glutamic acid R group, a lysine R group, a histidine R group, and an arginine R group.

42. The small molecule of claim 33, wherein R2 or R3 is —(CH2)j-COOH where j=1, 2, 3, or 4.

43. The small molecule of claim 33, wherein R2 or R3 is —(CH2)j-NH2 where j=1, 2, 3, 4, or 5, or —(CH2)j-NH—C(═NH)—NH2 where n=1, 2, 3 or 4.

44. The small molecule of claim 33, wherein at least one of n, x, and y, is 1 and P¹, P², P³ and P⁴ when present, are independently selected from the group consisting of polyethylene glycol (PEG), an acetyl, amide, 3 to 20 carbon alkyl groups, Fmoc, 9-fluoreneacetyl group, 1-fluorenecarboxylic group, 9-fluorenecarboxylic, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), Benzyloxymethyl (Bom), t-butoxycarbonyl (Boc), cyclohexyloxy (cHxO), t-butoxymethyl (Bum), t-butoxy (tBuO), t-Butyl (tBu), a propyl group, a butyl group, a pentyl group, a hexyl group, and trifluoroacetyl (TFA).

45. The small molecule of claim 44, wherein P1 when present and/or P2 when present are independently selected from the group consisting of Boc-, Fmoc-, and Nicotinyl-.

46. The small molecule of claim 44, wherein P3 when present and P4 are independently selected from the group consisting of tBu, and OtBu.

47. The small molecule of claim 45, wherein P3 when present and P4 are independently selected from the group consisting of tBu, and OtBu.

48. A pharmaceutical formulation, said formulation comprising a small molecule of claim 6 combined with a pharmacologically acceptable excipient.

49. The formulation of claim 48, wherein said excipient is an excipient suitable for oral administration to a mammal.

50. The formulation of claim 48, wherein said excipient is an excipient suitable for inhalation by a mammal.

51. The formulation of claim 48, wherein said formulation is provided as a unit dosage formulation.

52. The formulation of claim 48, wherein said formulation is provided as a time release formulation.

53. The formulation of claim 48, wherein said small molecule is provided in an amount sufficient to amelioriate a symptom of a pathology characterize by an inflammatory response.

54. The formulation of claim 53, wherein said pathology is selected from the group consisting of atherosclerosis, rheumatoid arthritis, lupus erythematous, polyarteritis nodosa, osteoporosis, Altzheimer's disease and a viral illnesses.

55. The formulation of claim 53, wherein said pathology is atherosclerosis.

56. The formulation of claim 48, wherein the formulation is formulated for administration by a route selected from the group consisting of oral administration, nasal administration, rectal administration, intraperitoneal injection, intravascular injection, subcutaneous injection, transcutaneous administration, inhalation administration, and intramuscular injection.

57. The formulation of claim 48, wherein the formulation further comprises one or more phospholipids.

58. A formulation for reducing cholesterol in a mammal, said formulation comprising one or more statins and/or Ezetimibe and a small molecule of claim 6.

59. The formulation of claim 58, wherein the small molecule and/or the statin or Ezetimibe are present in an effective dose.

60. The formulation of claim 58, wherein the effective amount of the statin is lower than the effective amount of the statin administered without the small molecule.

61. The formulation of claim 58, wherein the effective amount of the small molecule is lower than the effective amount of the small molecule administered without the statin.

62. The formulation of claim 58, wherein said statin comprises one or more statins selected from the group consisting of cerivastatin, atorvastatin, simvastatin, pravastatin, fluvastatin, lovastatin. rosuvastatin, and pitavastatin.

63. The formulation of claim 58, wherein said formulation is suitable for oral administration to a mammal.

64. The formulation of claim 58, wherein said formulation is provided as a unit dosage formulation.

65. The formulation of claim 58, wherein said formulation is provided as a time release formulation.

66. The formulation of claim 58, wherein said small molecule is provided in an amount sufficient to synergize the activity of said statin.

67. The formulation of claim 58, wherein the formulation is formulated for administration by a route selected from the group consisting of oral administration, inhalation, rectal administration, intraperitoneal injection, intravascular injection, subcutaneous injection, transcutaneous administration, inhalation administration, and intramuscular injection.

68. The formulation of claim 58, wherein the formulation further comprises one or more phospholipids.

69. A kit comprising:
a container containing one or more of the a small molecule of claim 6 through 17; and
instructional materials teaching the use of the small molecule(s) in the treatment of a pathology characterized by inflammation.

70. The kit of claim 69, wherein said pathology is a pathology selected from the group consisting of atherosclerosis, rheumatoid arthritis, lupus erythematous, polyarteritis nodosa, osteoporosis, Altzheimer's disease and a viral illnesses.

71. A method of mitigating one or more symptoms of atherosclerosis in a mammal, said method comprising administering to said mammal an effective amount of a small molecule according to claim 6.

72. The method of claim 71, wherein said small molecule is in a pharmaceutically acceptable excipient.

73. The method of claim 71, wherein said small molecule is administered in conjunction with a lipid and/or a statin.

74. The method of claim 71, wherein said small molecule is in a pharmaceutically acceptable excipient suitable for oral administration.

75. The method of claim 71, wherein said small molecule is administered as a unit dosage formulation.

76. The method of claim 71, wherein said administering comprises administering said small molecule by a route selected from the group consisting of oral administration, inhalation, rectal administration, intraperitoneal injection, intravascular injection, subcutaneous injection, transcutaneous administration, and intramuscular injection.

77. The method of claim 71, wherein said mammal is a mammal diagnosed as having one or more symptoms of atherosclerosis.

78. The method of claim 71, wherein said mammal is a mammal diagnosed as at risk for stroke or atherosclerosis.

79. The method of claim 71, wherein said mammal is a human.

80. The method of claim 71, wherein said mammal is non-human mammal.

81. A method of mitigating one or more symptoms of an inflammatory pathology in a mammal, said method comprising administering to said mammal an effective amount of one or more small molecules according to claim 6.

82. The method of claim 81, wherein said inflammatory pathology is a pathology selected from the group consisting of atherosclerosis, rheumatoid arthritis, lupus erythematous, polyarteritis nodosa, osteoporosis, Altzheimer's disease, multiple sclerosis, and a viral illnesses.

83. The method of claim 81, wherein said inflammatory pathology is atherosclerosis.

84. The method of claim 83, wherein said method further comprises administering an effective dose of a statin and/or Ezetimibe to said mammal.

85. The method of claim 81, wherein said small molecule is in a pharmaceutically acceptable excipient.

86. The method of claim 81, wherein said small molecule is administered in conjunction with a lipid and/or a statin.

87. The method of claim 81, wherein said small molecule is in a pharmaceutically acceptable excipient suitable for oral administration.

88. The method of claim 81, wherein said small molecule is administered as a unit dosage formulation.

89. The method of claim 81, wherein said administering comprises administering said peptide by a route selected from the group consisting of oral administration, inhalation, rectal administration, intraperitoneal injection, intravascular injection, subcutaneous injection, transcutaneous administration, and intramuscular injection.

90. The method of claim 81, wherein said mammal is a mammal diagnosed as at risk for stroke.

91. The method of claim 81, wherein said mammal is a human.

92. The method of claim 81, wherein said mammal is non-human mammal.

93. A method of enhancing the activity of a statin in a mammal, said method comprising coadministering with said statin an effective amount of one or more small molecules according to claim 6.

94. The method of claim 93, wherein said statin is selected from the group consisting of cerivastatin, atorvastatin, simvastatin, pravastatin, fluvastatin, lovastatin. rosuvastatin, and pitavastatin.

95. The method of claim 93, wherein said small molecule is administered simultaneously with said statin.

96. The method of claim 93, wherein said small molecule is administered before said statin.

97. The method of claim 93, wherein said small molecule is administered after said statin.

98. The method of claim 93, wherein said small molecule and/or said statin are administered as a unit dosage formulation.

99. The method of claim 93, wherein said administering comprises administering said small molecule and/or said statin by a route selected from the group consisting of oral administration, inhalation, rectal administration, intraperitoneal injection, intravascular injection, subcutaneous injection, transcutaneous administration, and intramuscular injection.

100. The method of claim 93, wherein said mammal is a mammal diagnosed as having one or more symptoms of atherosclerosis.

101. The method of claim 93, wherein said mammal is a mammal diagnosed as at risk for stroke or atherosclerosis.

102. The method of claim 93, wherein said mammal is a human.

103. The method of claim 93, wherein said mammal is non-human mammal.

104. A method of reducing or inhibiting one or more symptoms of osteoporosis in a mammal, the method comprising administering to the mammal one or more small molecules of claim 6, wherein the small molecule is administered in a concentration sufficient to reduce or eliminate one or more symptoms of osteoporosis.

105. The method of claim 104, wherein the small molecule is administered in a concentration sufficient to reduce or eliminate decalcification of a bone.

106. The method of claim 104, wherein the small molecule is administered in a concentration sufficient to induce recalcification of a bone.

107. The method of claim 104, wherein the small molecule is mixed with a pharmacologically acceptable excipient.

108. The method of claim 104, wherein the small molecule is mixed with a pharmacologically acceptable excipient suitable for oral administration to a mammal.
